SEQUENCE LISTING (4 sequences)

SEQ ID NO: 1 - DNA, 20 bp, Artificial Sequence
  Synthetic Forward FAS primer
tcagtacggagttggggaag

SEQ ID NO: 2 - DNA, 20 bp, Artificial Sequence
  Synthetic Forward FAS L primer
gcactttgggattctttcca

SEQ ID NO: 3 - DNA, 20 bp, Artificial Sequence
  Synthetic Reverse FAS primer
caggccttccaagttctgag

SEQ ID NO: 4 - DNA, 20 bp, Artificial Sequence
  Synthetic Reverse FAS L primer
cctccatttgtctggctcat

(19) United States
(12) Patent Application Publication
(10) Pub. No.: US 2017/0115293 A1
(43) Pub. Date: Apr. 27, 2017

(54) METHODS FOR PREDICTING THE RADIOSENSITIVITY OF A CANCER TUMOR AND METHODS OF TREATING CANCER

(71) Applicant: DUKE UNIVERSITY, Durham, NC (US)

(72) Inventors: Janet Horton, Durham, NC (US); Jen-Tsan Ashley Chi, Chapel Hill, NC (US); Sharareh Reihani, Chapel Hill, NC (US); Mark W. Dewhirst, Chapel Hill, NC (US)

(73) Assignee: DUKE UNIVERSITY, Durham, NC (US)

(21) Appl. No.: 15/331,416

(22) Filed: Oct. 21, 2016

Related U.S. Application Data
(60) Provisional application No. 62/244,281, filed on Oct. 21, 2015.

(51) Int. Cl. G01N 33/574; C07K 16/28; A61N 5/10; C12Q 1/68

(52) U.S. Cl. CPC G01N 33/57415; C12Q 1/6886; G01N 33/57484; C07K 16/2878; A61N 5/103; C12Q 2600/118; C12Q 2600/16; C12Q 2600/158; C07K 2317/75

(57) ABSTRACT

Methods for predicting the radiosensitivity of a cancer tumor and treating cancer in a subject are provided. In another aspect, methods of treating cancer in a subject are also provided. The methods may include administering to the subject radiation therapy based on the expression level of no more than 35, 30, 25, 20, 15, 10, or 5 biomarkers in a tumor sample from the subject. The methods of treating cancer in a subject may also include administering a therapeutically effective amount of a FAS agonist and/or a TNFRSF10B agonist to the subject, and administering radiation therapy to the subject.

### CROSS-REFERENCE TO RELATED APPLICATIONS

This patent application claims the benefit of priority of U.S. Provisional Patent Application No. 62/244,281, filed Oct. 21, 2015, which is incorporated herein by reference in its entirety.

### STATEMENT REGARDING FEDERALLY SPONSORED RESEARCH

This invention was made with United States government support awarded by the National Institute of Health grant number K12HD043446. The United States has certain rights in this invention.

### INTRODUCTION

Delivery of radiation therapy in the treatment of cancer is currently based on historic data that predated modern molecular techniques and an understanding of cancer subtype-specific biology. For example, with respect to breast cancer, for almost 300,000 women in the U.S. diagnosed with breast cancer each year, the “one size fits all” radiotherapy approach is a routine part of multidisciplinary breast cancer care. However, clinical data are accumulating to support the idea that radiation response can be linked to breast cancer phenotype and that the more biologically aggressive phenotypes may display greater radiation resistance. Though radiotherapy is generally efficacious, the variation in subtype specific RT response suggests that current uniform practice is inadequate on two fronts: i) patients with more radiosensitive tumors may receive unnecessarily high radiation doses and radiation-related tissue damage; ii) patients with more radioresistant tumors may have an unacceptably high rate of morbidity and mortality due to higher rates of tumor recurrence. Thus, there is a need in the art for methods of predicting the responsiveness of a cancer in a subject to a radiation therapy and methods of treating cancer using such methods such that optimal doses of radiation may be administered to cancer patients.

### SUMMARY

In one aspect, methods for predicting the radiosensitivity of a cancer tumor and treating cancer in a subject are provided. The methods may include i) obtaining a tumor sample from a subject, and ii) measuring the expression level of no more than 35, 30, 25, 20, 15, 10, or 5 biomarkers in the sample. The biomarkers measured in the tumor sample may include at least one biomarker selected from FAS, TNFRSF10B, CD48, LST1, LY86, CCR1, LY96, AIF1, TNFSF13B, TP5313 (p53-inducible protein 3) and caspase 9 or any combination thereof. In some embodiments, the biomarkers include FAS, TNFRSF10B, or both.

In another aspect, methods of treating cancer in a subject are also provided. The methods may include administering to the subject radiation therapy based on the expression level of no more than 35, 30, 25, 20, 15, 10, or 5 biomarkers in a tumor sample from the subject.

In a further aspect, the methods of treating cancer in a subject may also include administering a therapeutically effective amount of a FAS agonist and/or a TNFRSF10B agonist to the subject, and administering radiation therapy to the subject. The administration of the radiation therapy may be before, during, or after administration of the FAS agonist and/or a TNFRSF10B agonist to the subject.

### BRIEF DESCRIPTION OF THE DRAWINGS

FIG. 1 shows the response to radiation in paired pre- and postirradiation breast tumor samples and a panel of diverse breast cancer cell lines. Panel A: Overview schematic of research plan. Panel B: Principal component analysis on human breast cancers suggests that gene expression profiles after irradiation are significantly and consistently distinct from that noted prior to radiation. Panel C: Induction of gene expression dominates repression in these samples. The 100 genes with the most significant change in response to radiation are illustrated in green. Panel D: 16 cell lines representing diverse biologic phenotypes: luminal HER2—(MCF7, T47D, ZR751, CAMA-1), luminal-HER2 [symbol] (BT474, SKBR3, AU565), HER2 [symbol] (HCC1954) and basal (SUM149, SUM159, MDA-MB-231, MCF10A, MCF12A, BT549, HBL100 and DKAT) were evaluated for response to a single dose of 5 Gy. Four of the cell lines (3 HER2—luminal lines: MCF7, ZR751, T47D, and 1 basal: HBL100) had a large number of genes with significant induction or repression after irradiation. In contrast, the remaining twelve basal and HER2 [symbol] luminal cell lines revealed a striking absence of response to radiation. Panel E: Fourteen genes associated with 27 probe sets were identified as having significant Q values for differential expression in both the human data and in the cell line data (Q, 0.002). FAS is significantly induced with an effect conserved across multiple probe sets.

FIG. 2 shows patterns of FAS induction in response to radiation treatment in human breast tumor samples. Panel A: FAS response to radiation in the paired pre- and postirradiation gene expression samples from our clinical trial (Q¼ 7.42 E-10). Black circles represent preirradiation expression levels and red circles represent postirradiation expression. Panel B: FAS immunohistochemistry (IHC) in two selected patients (panels A and B) pre- (left side) and postirradiation (right side). Photos were taken at 2003. Panel C: Mean FAS IHC scores nearly doubled (P ¼ 0.004) when including all interpretable patient samples preirradiation (n ¼ 27) and postirradiation (n ¼ 20). Panel D: Sixteen of 32 patients had paired pre- and postirradiation FAS IHC. Six of 16 patients showed significant FAS induction (change in histoscore 0.100) and 4 were in the highest dose cohort (21 Gy) suggesting a doseresponse relationship.

FIG. 3 shows the FAS response to radiation in breast cancer cell lines. Panel A: FAS induction is noted in 3 of the 4 radiation-responsive cell lines (black circles represent preirradiation gene expression; red circles represent postirradiation gene expression; arrows represent statistically significant findings). This is not seen in the nonresponsive cell lines, despite highly variable baseline levels of FAS. Panel B: qPCR confirms differential patterns of FAS response to radiation in phenotypically distinct cell lines (*P, 0.05). Panel C: At the protein level, FAS protein is again increased in the radiation-responsive (MCF7, ZR751) group and does not significantly change in the nonresponsive (MDAMB231, SUM159) cell lines. All results shown are for 24 h after a single dose of 5 Gy.

FIG. 4 shows the effects of radiation on FAS trafficking. MCF7 and SUM159 cells were treated with a 0 and 5 Gy dose of radiation and stained with anti-FAS Ab 24 h after irradiation. FAS expression was analyzed by ImageStream flow cytometry. Panel A: Radiation exposure increased the total FAS intensity in the radiation-responsive cell line MCF7, but had little effect on the radiation-nonresponsive cell line, SUM159. Panel B: Images of FAS staining in SUM159 cells demonstrated that FAS was expressed both on the cell surface and in the cytoplasm. Panel C: To evaluate the localization of FAS, we generated masks for cytoplasmic and membrane FAS to quantify their intensity. Panels D and E: In response to radiation, both cytoplasmic and membrane FAS increased in the MCF7 cell line. In contrast, SUM159 had high levels of membrane and cytoplasmic FAS before and after irradiation.

FIG. 5 shows the effect of FAS modulation on radiation response. Panel A: FAS was silenced using shRNA in the radiation-responsive MCF7 cells. Panel B: Clonogenic assays revealed an increase in radiation resistance at 1 and 2 Gy dose levels in the absence of FAS (*P, 0.05). At 4 Gy, survival was equivalent suggesting activation of pathways other than FAS-mediated apoptosis, possibly dose-dependent, in this radiation-sensitive cell line. Panel C: In contrast, stimulation of FAS increased sensitivity to radiation in the radiation-nonresponsive cell line, SUM159. The dose to achieve a surviving fraction of 0.1 was approximately 8 Gy in the control cells and 3.25 Gy in the CH11-treated SUM159 cells, yielding a dose-modifying factor of 2.5. Panel D: Enhanced radiation sensitivity occurred despite a lack of significant change in FAS expression levels after pre-treatment with CH11, a FAS activating antibody (0.1 lg/ml).

FIG. 6 shows FAS signaling in basal cell lines with variable levels of baseline FAS. After pre-treatment with CH11, a FAS activating antibody (0.1 1g/ml), we observed induction of apoptosis proteases caspases 3, 8 and 9, as well as c-PARP in two cell lines with high baseline levels of FAS, SUM159 and HCC1954, suggesting an intact apoptosis pathway. In contrast, this was not observed in the two cell lines with low levels of baseline FAS, MDAMB231 and AU565.

FIG. 7 shows the impact of FAS overexpression on radiation response phenotype in basal cell lines with low levels of baseline FAS. Panel A: FAS overexpressing MDMBA231 and AU565 cell lines treated with CH11 demonstrate induction of the apoptosis proteases, caspases 3, 8 and 9 and c-PARP. Panel B: Reintroduction of FAS signaling enhanced radiation response in both basal cell lines.

FIG. 8 shows radiation-induced FAS expression patterns in SUM159 cell line.

FIG. 9 shows FAP1 and FAS co-localization.

FIG. 10 shows the effects of radiation on FAS ligand expression and cellular location in response to 0 and 5 Gy irradiation.

FIG. 11 shows the relationship between TP53 and FAS induction.

FIG. 12 shows the relationship between FAS and RSI in TCGA database.

FIG. 13 shows preoperative radiation to the intact tumor. Sagittal view (A) of a prone treatment planning MRI (left) and CT (right). (B) Sagittal (left) and axial (right) treatment planning images with dose distribution in the same patient.

FIG. 14 shows cosmetic outcomes for single-dose, definitive pre-operative partial breast irradiation. (A) Patient and physician-reported cosmetic outcomes in patients treated with preoperative radiation only. (B) Two women with more than 3 years of follow-up. Red arrows highlight the region of lumpectomy.

FIG. 15 shows paired pre and post-radiation MRI images demonstrate evidence of increased vascular permeability. (A) Pre- (left) and post-treatment (right) DCE-MRI images. (B) Pre- (left) and post-treatment (right) images highlighting contrast agent distribution with the planning target volume.

FIG. 16 shows changes in gene expression following radiation in early-stage favorable breast tumors (ER+). (A) Principal component analysis suggests that gene expression profiles following radiation are significantly and consistently distinct from that noted prior to radiation. (B) The primary effect of increasing dose is to enhance, rather than repress, gene expression in the subset of 27 genes experiencing significant and dose-related change with radiation. (C) The impact of radiation on relative gene expression increases (n=24 of 27) with each incremental increase in dose. (D) The cohort of genes demonstrating significant dose-response is enriched for modulators of immunity and inflammation.

FIG. 17 shows the differential gene expression response to radiation in a panel of 16 breast cancer cell lines. A) In response to RT, four of the cell lines, three luminal, (MCF7, ZR751, T47D) had a large number of differentially expressed genes. In contrast, the remaining largely basal and HER2+ cell lines had minimal changes in gene expression following radiation. B and C) The effect of radiation on the expression levels of TNFRSF10B and FAS is significantly different in the responsive vs. non responsive cell lines. (Black circles are pre-RT and red circles are postRT).

### DETAILED DESCRIPTION

Although a standardized approach to radiotherapy has been used to treat breast cancer, regardless of subtype (e.g., luminal, basal), recent clinical data suggest that radiation response may vary significantly among subtypes. To develop improved methods of diagnosing and treating cancers with radiation therapy, the present inventors, in the non-limiting Examples, utilized RNA samples for microarray analysis from two sources: 1. Paired pre- and postirradiation breast tumor tissue from 32 early-stage breast cancer patients treated in our unique preoperative radiation Phase I trial; and 2. Sixteen biologically diverse breast tumor cell lines exposed to 0 and 5 Gy irradiation. The transcriptome response to radiation exposure was derived by comparing gene expression in samples before and after irradiation. Genes with the highest coefficient of variation were selected for further evaluation and validated at the RNA and protein level. Gene editing and agonistic antibody treatment were performed to assess the impact of gene modulation on radiation response. Gene expression in our cohort of luminal breast cancer patients was distinctly different before and after irradiation. Further, two distinct patterns of gene expression were observed in our biologically diverse group of breast cancer cell lines pre- versus postirradiation. Cell lines that showed significant change after irradiation were largely luminal subtype, while gene expression in the basal and HER2+ cell lines was minimally impacted. The 100 genes with the most significant response to radiation in patients were identified and analyzed for differential patterns of expression in the radiation-responsive versus nonresponsive cell lines. Fourteen genes were identified as significant, including FAS, a member of the tumor necrosis factor receptor family known to play a critical role in programmed cell death. Modulation of FAS in breast cancer cell lines altered radiation response phenotype and enhanced radiation sensitivity in radioresistant basal cell lines. The present inventors' findings suggest, in part, that cell-type specific, radiation-induced FAS contributes to subtype specific breast cancer radiation response and that activation of FAS pathways may be exploited for biologically tailored radiotherapy.

Methods for predicting the radiosensitivity of a cancer tumor and treating cancer in a subject are provided. The methods may include i) obtaining a tumor sample from a subject, and ii) measuring the expression level of no more than 35, 30, 25, 20, 15, 10, or 5 biomarkers in the sample.

As used herein, a “tumor sample” is a sample containing at least one cell taken from or around a cancer tumor by, for example, a biopsy or obtained after a tumor is removed from the subject. In accordance with the present methods, the cancer may be any cancer including, without limitation, breast, colorectal, pancreatic, liver, esophageal, gastric, kidney, small bowel, cholangiocarcinoma, lung, head and neck, thyroid, melanoma, breast, renal, bladder, ovarian, cervical, uterine, prostate, lymphomas, leukemias, neuroendocrine, glioblastoma or any other form of brain cancer. Preferably, the cancer is breast cancer and the tumor sample is a breast cancer tumor. The cancer may be an “early-stage” breast cancer. The breast cancer may be an ER+ cancer.

The terms “subject” and “patient” are used interchangeably herein and refer to both human and non-human animals. The term “non-human animals” of the disclosure includes all vertebrates, e.g., mammals and non-mammals, such as nonhuman primates, sheep, dog, cat, horse, cow, chickens, amphibians, reptiles, and the like. Preferably, the subject is a human patient. More preferably, the subject is a human patient diagnosed with cancer or undergoing, or about to undergo or has undergone, a cancer radiation regimen.

As used herein, a “biomarker” is a polynucleotide or protein whose level of expression in a sample is indicative of a condition. In the Examples, the biomarkers are measured by assessing the expression levels of mRNA transcripts and/or proteins encoded by genes. In some embodiments, the expression level of the biomarker is the protein expression level. In some embodiments, the expression level of the biomarker is the mRNA expression level. These expression levels have, for example, been found to correlate with the radiosensitivity of a cancer tumor.

Biomarker expression in some instances may be normalized against the expression levels of all proteins or RNA transcripts in the sample, or against a reference set of proteins or RNA transcripts in the sample.

Fragments and variants of biomarker mRNA transcripts and proteins are also encompassed by the present invention. A “fragment” is intended to refer to a portion of the polynucleotide or a portion of the amino acid sequence and hence protein encoded thereby. Polynucleotides that are fragments of a biomarker nucleotide sequence generally comprise at least 10, 15, 20, 50, 75, 100, 150, 200, 250, 300, 350, 400, 450, 500, 550, 600, 650, 700, 800, 900, 1,000, 1,200, or 1,500 contiguous nucleotides, or up to the number of nucleotides present in a full-length biomarker polynucleotide disclosed herein. A fragment of a biomarker polypeptides will generally encode at least 15, 25, 30, 50, 100, 150, 200, or 250 contiguous amino acids, or up to the total number of amino acids present in a full-length biomarker protein of the invention. “Variant” is intended to mean substantially similar sequences. Generally, variants of a particular biomarker of the invention will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that biomarker as determined by sequence alignment programs.

Any methods available in the art for detecting expression of biomarkers are encompassed herein. The expression of a biomarker of the invention can be detected on a nucleic acid level (e.g., as an mRNA transcript) or a protein level. “Measuring the expression level” means determining the quantity or presence of a protein or its RNA transcript for at least one of the biomarkers disclosed herein. Thus, “measuring the expression level” encompasses instances where a biomarker is determined not to be expressed, not to be detectably expressed, expressed at a low level, expressed at a normal level, or overexpressed. The expression level may be measured relative to a control.

Methods suitable for measuring, detecting, or determining the expression levels of biomarkers are known to those of skill in the art and include, but are not limited to, ELISA, immunofluorescence, FACS analysis, Western blot, magnetic immunoassays, and both antibody-based microarrays and non-antibody-based microarrays. In the past, the gold standard for detection of growth factors and cytokines in blood was the use of ELISAs; however, multiplex technology offers an attractive alternative approach for cytokine and growth factor analysis. The advantages of multiplex technology compared to traditional ELISA assays are conservation of patient sample, increased sensitivity, and significant savings in cost, time and labor.

Several multiplex platforms currently exist. The Luminex bead-based systems are the most established, being used to detect circulating cytokines and growth factors in both mice and humans. This method is based on the use of microparticles that have been pre-coated with specific antibodies. These particles are then mixed with sample and the captured analytes are detected using specific secondary antibodies. This allows for up to 100 different analytes to be measured simultaneously in a single microplate well. The advantages of this flow cytometry-based method compared to traditional ELISA assays are in the conservation of patient samples as well as significant savings in terms of cost and labor. An alternative, plate-based system is produced by Meso Scale Discovery (MSD). This system utilizes its proprietary Multi-Array® and Multi-Spot® microplates with electrodes directly integrated into the plates. This enables the MSD system to have ultra-sensitive detection limits, high specificity, and low background signal. Another plate-based multiplex system is the SearchLight Plus CCD Imaging System produced by Aushon Biosystems. This novel multiplexing technology allows for the measurement of up to 16 different analytes simultaneously in a single microplate well. The assay design is similar to a sandwich ELISA where the capture antibodies are pre-spotted into individual wells of a 96-well plate. Samples or standards are added which bind to the specific capture antibodies and are detected using Aushon's patented SuperSignal ELISA Femto Chemiluminescent Substrate.

Methods for detecting expression of the biomarkers described herein are not limited to protein expression. Gene expression profiling including methods based on hybridization analysis of polynucleotides, methods based on sequencing of polynucleotides, immunohistochemistry methods, and proteomics-based methods may also be used. The most commonly used methods known in the art for the quantification of mRNA expression in a sample include northern blotting and in situ hybridization (Parker and Barnes, Methods Mol. Biol. 106:247-83, 1999), RNAse protection assays (Hod, Biotechniques 13:852-54, 1992), PCR-based methods, such as reverse transcription PCR(RT-PCR) (Weis et al., TIG 8:263-64, 1992), including real time quantitative PCR and array-based methods (Schena et al., Science 270:467-70, 1995). Alternatively, antibodies may be employed that can recognize specific duplexes, including DNA duplexes, RNA duplexes, and DNA-RNA hybrid duplexes, or DNA-protein duplexes. Representative methods for sequencing-based gene expression analysis include Serial Analysis of Gene Expression (SAGE) and gene expression analysis by massively parallel signature sequencing.

In the methods described herein the expression level of at least one biomarker described herein in a sample from the subject is determined using any one of the detection methods described herein. Then the level in the sample from the subject is compared to a reference level of the biomarker or a control. The “reference level” may be determined empirically such as it was in the Examples, by comparison to the levels found in a set of samples from cancer patients treated as described with known clinical outcomes for the patients. Alternatively, the reference level may be a level of the biomarker found in samples, such as plasma samples or non-cancerous cells such as adjacent non-cancerous cells from the same subject, which becomes a standard and can be used as a predictor for new samples.

In accordance with the present invention, the biomarkers measured in the tumor sample may include at least one biomarker selected from FAS, TNFRSF10B, CD48, LST1, LY86, CCR1, LY96, AIF1, TNFSF13B, TP5313 (p53-inducible protein 3) and caspase 9 or any combination thereof. In some embodiments, the biomarkers include FAS, TNFRSF10B, or both. In one embodiment, the biomarkers include at least FAS, TNFRSF10B, CD48, LST1, LY86, CCR1, LY96, AIF1, TNFSF13B, TP5313 (p53-inducible protein 3) and caspase 9.

The methods disclosed herein may further include administering radiation therapy to the subject. The radiation therapy may be administered to the subject prior to obtaining the tumor sample from the subject. Alternatively, the radiation therapy may be administered to the subject after measuring the expression level of the biomarkers in the sample. Alternatively, tumor samples may be obtained both before and after administration of radiation therapy and in some embodiments the expression levels of the biomarkers are compared in the cancer cells before and after radiation therapy. An increase in expression of the biomarkers after radiation therapy as compared to expression of the same biomarker before radiation therapy is indicative of radiation responsiveness of the cancer. In subjects with a radiation responsive cancer, the radiation therapy may be deemed to be a sufficient dosage to treat the cancer. In subjects with no change in expression of the biomarker after treatment with the radiation therapy, radiation therapy may be repeated or repeated with an increased dose of radiation if the biomarkers are expressed in the tumor cells. If the tumor cells lack expression or have low levels of expression of the biomarkers in the tumor sample, an alternative treatment should be pursued as these tumor cells are likely to be radiation resistant and a different form of therapy should be added to the treatment regimen. The radiation therapy may include one or more doses of between 1 Gy and 30 Gy. Suitably, the radiation therapy includes a single fraction dose of 12, 15, 18, 20, 21, 23, 25, or 28 Gy.

Methods of treating cancer in a subject are also provided. The methods may include administering to the subject radiation therapy based on the expression level of no more than 35, 30, 25, 20, 15, 10, or 5 biomarkers in a tumor sample from the subject.

The methods of treating cancer in a subject may also include administering a therapeutically effective amount of a FAS agonist and/or a TNFRSF10B agonist to the subject, and administering radiation therapy to the subject. The administration of the radiation therapy may be before, during, or after administration of the FAS agonist and/or a TNFRSF10B agonist to the subject. In embodiments where both the FAS agonist and the TNFRSF10B agonist are administered they may be administered concurrently or at different times, particularly in relation to the administration of the radiation therapy.

“Treating cancer” includes, without limitation, reducing the number of cancer cells or the size of a tumor in the subject, reducing progression of a cancer to a more aggressive form (i.e. maintaining the cancer in a form that is susceptible to a therapeutic agent), reducing proliferation of cancer cells or reducing the speed of tumor growth, killing of cancer cells, reducing metastasis of cancer cells or reducing the likelihood of recurrence of a cancer in a subject. Treating a subject as used herein refers to any type of treatment that imparts a benefit to a subject afflicted with cancer or at risk of developing cancer or facing a cancer recurrence. Treatment includes improvement in the condition of the subject (e.g., in one or more symptoms), delay in the progression of the disease, delay in the onset of symptoms or slowing the progression of symptoms, etc.

As used herein, a “FAS agonist” is any agent capable of partially or fully activating one or more of the biological activities of a FAS protein including, without limitation, a polypeptide, a polynucleotide, or a small molecule. A FAS agonist may function in a direct or indirect manner. For example, the FAS agonist may directly bind to a FAS protein, thus partially or fully activating one or more biological activities of a FAS protein, in vitro or in vivo. The FAS agonist may also function indirectly by (1) interacting with (e.g., activating, inducing, blocking or inhibiting) another molecule that can bind to FAS, such as a FAS ligand, or (2) modulating or affecting the expression (i.e, transcription or translation) of a FAS protein in a cell.

Mammalian FAS proteins are members of the tumor necrosis factor (TNF) receptor superfamily, a family of transmembrane receptors. FAS proteins have been shown to be an important mediator of apoptotic cell death, as well as being involved in inflammation.

As used herein, a “TNFRSF10B Agonist” is any agent capable of partially or fully activating one or more of the biological activities of a TNFRSF10B protein including, without limitation, a polypeptide, a polynucleotide, or a small molecule. A TNFRSF10B Agonist may function in a direct or indirect manner. For example, the TNFRSF10B Agonist may directly bind to a TNFRSF10B protein, thus partially or fully activating one or more biological activities of a TNFRSF10B protein, in vitro or in vivo. The TNFRSF10B Agonist may also function indirectly by (1) interacting with (e.g., activating, inducing, blocking or inhibiting) another molecule that can bind to TNFRSF10B or (2) modulating or affecting the expression (i.e, transcription or translation) of a TNFRSF10B protein in a cell.

Mammalian TNFRSF10B proteins (also called TRAIL R2, DRS, and TRICK 2) are a type 1, TNF R family, membrane protein which is a receptor for TRAIL (APO2 ligand). TNFRSF10B proteins contain an extracellular cysteine-rich domain, a transmembrane domain and a cytoplasmic death domain.

FAS and TNFRSF10B proteins may be any of the FAS and TNFRSF10B proteins found in any mammal including, without limitation, humans or domesticated animals such as dogs, cats, horses, cows, pigs, mice, or rats. Suitably, the FAS and TNFRSF10B agonists disclosed herein activate the human FAS and TNFRSF10B proteins, respectively.

The FAS and/or TNFRSF10B agonists may be polypeptides including, without limitation, a peptide or an antibody. As used herein, the term “antibody” is used in the broadest sense used in the art to refer to polypeptide affinity agents based on antibodies. For example, the antibody may include a polyclonal antibody, a monoclonal antibody, a single chain antibody, or antibody fragments such as Fab, Fab′, F(ab′)₂, Fv fragments, diabodies, linear antibodies, or multispecific antibodies formed from antibody fragments. The antibody may be chimeric, humanized, or fully human. The antibody may be any one of the five known major classes of immunoglobulins including IgA, IgD, IgE, IgG, and IgM. In some embodiments, the FAS and/or TNFRSF10B agonists may be an antibody that is capable of binding a FAS and/or TNFRSF10B protein and thereby partially or fully activating one or more of the biological activities of a FAS and/or TNFRSF10B protein. In the non-limiting Examples, the inventors use the CH 11 FAS agonistic antibody which was obtained from Millipore, cat #05-201 and the TNFRSF10B agonistic antibody was obtained from R&D systems, cat #MAB63 1.

Peptides useful as FAS and/or TNFRSF10B agonists may be identified using techniques well-known in the art such as phage display.

Aptamers are polynucleotides (e.g., ssDNA or ssRNA) that bind to a specific target molecule. In some embodiments, the FAS and/or TNFRSF10B agonists may be an aptamer that is capable of binding a FAS and/or TNFRSF10B protein and thereby partially or fully activating one or more of the biological activities of a FAS and/or TNFRSF10B protein.

The FAS and/or TNFRSF10B agonists may be a small molecule. The small molecule may be chemical molecule having a molecular weight below about 2500 Daltons, 2000 Daltons, 1000 Daltons, or 500 Daltons.

As used herein, an “effective amount” or a “therapeutically effective amount” as used herein means the amount of a composition that, when administered to a subject for treating a state, disorder or condition is sufficient to effect a treatment (as defined above). The therapeutically effective amount will vary depending on the compound, formulation or composition, the disease and its severity and the age, weight, physical condition and responsiveness of the subject to be treated.

The FAS and/or TNFRSF10B agonists described herein may be administered by any means known to those skilled in the art, including, without limitation, intravenously, intra-tumoral, intra-lesional, intradermal, topical, intraperitoneal, intramuscular, parenteral, subcutaneous and topical administration Thus the compositions may be formulated as an injectable, topical, ingestible, or suppository formulation. Administration of the FAS and/or TNFRSF10B agonists to a subject in accordance with the present invention may exhibit beneficial effects in a dose-dependent manner. Thus, within broad limits, administration of larger quantities of the compositions is expected to achieve increased beneficial biological effects than administration of a smaller amount. Moreover, efficacy is also contemplated at dosages below the level at which toxicity is seen.

It will be appreciated that the specific dosage of FAS and/or TNFRSF10B agonist administered in any given case will be adjusted in accordance with the composition or compositions being administered, the volume of the composition that can be effectively delivered to the site of administration, the disease to be treated or inhibited, the condition of the subject, and other relevant medical factors that may modify the activity of the compositions or the response of the subject, as is well known by those skilled in the art. For example, the specific dose of a FAS and/or TNFRSF10B agonist for a particular subject depends on age, body weight, general state of health, diet, the timing and mode of administration, the rate of excretion, medicaments used in combination and the severity of the particular disorder to which the therapy is applied. Dosages for a given patient can be determined using conventional considerations, e.g., by customary comparison of the differential activities of the compositions described herein and of a known agent, such as by means of an appropriate conventional pharmacological protocol. The compositions can be given in a single dose schedule, or in a multiple dose schedule.

The maximal dosage of a FAS and/or TNFRSF10B agonist for a subject is the highest dosage that does not cause undesirable or intolerable side effects. The number of variables in regard to an individual treatment regimen is large, and a considerable range of doses is expected. The route of administration will also impact the dosage requirements. It is anticipated that dosages of the compositions will treat cancer by, for example, by reducing tumor size or decreasing the rate of tumor growth by least 10%, 20%, 30%, 40%, 50%, 60%, 70%, 80%, 90%, 100% or more as compared to no treatment.

The effective dosage amounts of a FAS and/or TNFRSF10B agonists refer to total amounts administered, that is, if more than one composition is administered, the effective dosage amounts of a FAS and/or TNFRSF10B agonist corresponds to the total amount administered. The compositions can be administered as a single dose or as divided doses. For example, the composition may be administered two or more times separated by 4 hours, 6 hours, 8 hours, 12 hours, a day, two days, three days, four days, one week, two weeks, or by three or more weeks.

Suitable dosage ranges for a FAS and/or TNFRSF10B agonist may be of the order of several hundred micrograms of the agent with a range from about 0.001 to 10 mg/kg/day, preferably in the range from about 0.01 to 1 mg/kg/day. Precise amounts of a FAS and/or TNFRSF10B agonist required to be administered depend on the judgment of the practitioner and may be peculiar to each subject. It will be apparent to those of skill in the art that the therapeutically effective amount of the compositions and pharmaceutical compositions described herein will depend, inter alia, upon the administration schedule, the unit dose of agent administered, whether the composition is administered in combination with other therapeutic agents, the status and health of the recipient, and the therapeutic activity of the particular composition.

The present disclosure is not limited to the specific details of construction, arrangement of components, or method steps set forth herein. The compositions and methods disclosed herein are capable of being made, practiced, used, carried out and/or formed in various ways that will be apparent to one of skill in the art in light of the disclosure that follows. The phraseology and terminology used herein is for the purpose of description only and should not be regarded as limiting to the scope of the claims. Ordinal indicators, such as first, second, and third, as used in the description and the claims to refer to various structures or method steps, are not meant to be construed to indicate any specific structures or steps, or any particular order or configuration to such structures or steps. All methods described herein can be performed in any suitable order unless otherwise indicated herein or otherwise clearly contradicted by context. The use of any and all examples, or exemplary language (e.g., “such as”) provided herein, is intended merely to facilitate the disclosure and does not imply any limitation on the scope of the disclosure unless otherwise claimed. No language in the specification, and no structures shown in the drawings, should be construed as indicating that any non-claimed element is essential to the practice of the disclosed subject matter. The use herein of the terms “including,” “comprising,” or “having,” and variations thereof, is meant to encompass the elements listed thereafter and equivalents thereof, as well as additional elements. Embodiments recited as “including,” “comprising,” or “having” certain elements are also contemplated as “consisting essentially of” and “consisting of” those certain elements.

Recitation of ranges of values herein are merely intended to serve as a shorthand method of referring individually to each separate value falling within the range, unless otherwise indicated herein, and each separate value is incorporated into the specification as if it were individually recited herein. For example, if a concentration range is stated as 1% to 50%, it is intended that values such as 2% to 40%, 10% to 30%, or 1% to 3%, etc., are expressly enumerated in this specification. These are only examples of what is specifically intended, and all possible combinations of numerical values between and including the lowest value and the highest value enumerated are to be considered to be expressly stated in this disclosure. Use of the word “about” to describe a particular recited amount or range of amounts is meant to indicate that values very near to the recited amount are included in that amount, such as values that could or naturally would be accounted for due to manufacturing tolerances, instrument and human error in forming measurements, and the like. All percentages referring to amounts are by weight unless indicated otherwise.

No admission is made that any reference, including any non-patent or patent document cited in this specification, constitutes prior art. In particular, it will be understood that, unless otherwise stated, reference to any document herein does not constitute an admission that any of these documents forms part of the common general knowledge in the art in the United States or in any other country. Any discussion of the references states what their authors assert, and the applicant reserves the right to challenge the accuracy and pertinence of any of the documents cited herein. All references cited herein are fully incorporated by reference in their entirety, unless explicitly indicated otherwise. The present disclosure shall control in the event there are any disparities between any definitions and/or description found in the cited references.

Unless otherwise specified or indicated by context, the terms “a”, “an”, and “the” mean “one or more.” For example, “a protein” or “an RNA” should be interpreted to mean “one or more proteins” or “one or more RNAs,” respectively.

The following examples are meant only to be illustrative and are not meant as limitations on the scope of the invention or of the appended claims.

### EXAMPLES

### Example 1

### FAS Death Receptor: a Breast Cancer Subtype-Specific Radiation Response Biomarker and Potential Therapeutic Target

### Abstract

Although a standardized approach to radiotherapy has been used to treat breast cancer, regardless of subtype (e.g., luminal, basal), recent clinical data suggest that radiation response may vary significantly among subtypes. We hypothesized that this clinical variability may be due, in part, to differences in cellular radiation response. In this study, we utilized RNA samples for microarray analysis from two sources: 1. Paired pre- and postirradiation breast tumor tissue from 32 early-stage breast cancer patients treated in our unique preoperative radiation Phase I trial; and 2. Sixteen biologically diverse breast tumor cell lines exposed to 0 and 5 Gy irradiation. The transcriptome response to radiation exposure was derived by comparing gene expression in samples before and after irradiation. Genes with the highest coefficient of variation were selected for further evaluation and validated at the RNA and protein level. Gene editing and agonistic antibody treatment were performed to assess the impact of gene modulation on radiation response. Gene expression in our cohort of luminal breast cancer patients was distinctly different before and after irradiation. Further, two distinct patterns of gene expression were observed in our biologically diverse group of breast cancer cell lines pre- versus post irradiation. Cell lines that showed significant change after irradiation were largely luminal subtype, while gene expression in the basal and HER2+ cell lines was minimally impacted. The 100 genes with the most significant response to radiation in patients were identified and analyzed for differential patterns of expression in the radiation-responsive versus nonresponsive cell lines. Fourteen genes were identified as significant, including FAS, a member of the tumor necrosis factor receptor family known to play a critical role in programed cell death. Modulation of FAS in breast cancer cell lines altered radiation response phenotype and enhanced radiation sensitivity in radioresistant basal cell lines. Our findings suggest that cell-type specific, radiation-induced FAS contributes to subtype specific breast cancer radiation response and that activation of FAS pathways may be exploited for biologically tailored radiotherapy.

### Introduction

Radiotherapy is a routine component of multidisciplinary care for patients with breast cancer. However, despite increasingly detailed knowledge of breast tumor biology, the daily prescription of radiotherapy has remained relatively constant over many decades. It is now well known that breast tumors are composed of a heterogeneous group of distinct subtypes with characteristic clinical outcomes and patterns of gene expression (1). Moreover, it has been demonstrated repeatedly that breast cancer subtypes vary in their response to chemotherapy (2-4). Although less is known about the relationship between radiation response and breast cancer subtypes, previously reported clinical data have suggested that the more biologically aggressive phenotypes may display greater radiation resistance (5-7). In an analysis of 2,985 breast tumors, Voduc et al. noted clear variation in locoregional recurrence risk. These inherent subtype-specific differences in locoregional relapse were quite clear in patients treated with mastectomy, largely without radiation therapy. However, the subtype-specific patterns of locoregional recurrence persisted even in the breast conservation setting where radiation was uniformly delivered, suggesting that the more biologically aggressive subtypes are also more resistant to radiotherapy (7). Mechanisms underlying a differential response to radiotherapy in breast cancer subtypes are not well understood. Amundson et al. previously characterized a 60-cell-line panel from the National Cancer Institute for survival, apoptosis and gene expression in response to radiation exposure. This data suggested that the basal gene expression pattern was superior to radiation response signatures in discriminating radiosensitive from radioresistant cell lines. However, this panel incorporated only a few breast cancer cell lines and not all subtypes were included (8). Helland et al. (9) reported on changes in gene expression using pre- and postirradiation tumor biopsies from 19 patients with breast cancer. They identified four genes with consistent induction, two of which were involved in DNA repair and cell cycle control. However, all patients in this series had locally advanced or metastatic tumors, a setting in which inherent biologic differences may not be as clear.

Our overall hypothesis is that clinical differences in locoregional control among breast cancer subtypes are associated with unique radiation response gene expression profiles. Because breast cancer radiotherapy is typically delivered almost exclusively in the postoperative setting (10), opportunities to study this response are rare. However, we have generated a unique cohort of paired pre- and postirradiation samples from our clinical trial of patients with biologically favorable early-stage breast cancer treated with preoperative radiation therapy. To the best of our knowledge, our group was the first to propose delivery of radiation therapy prior to surgical resection, and we have since optimized this technique (11). In the current study, using our patient samples and a biologically diverse panel of breast tumor cell lines, we examined the mechanisms underlying observed biological diversity in breast cancer radiation response and looked for subtype-specific patterns of radiation response that could act as a starting point to link radiation response phenotype to potentially targetable biologic pathways (12).

### Methods

### Breast Cancer Patients

Patients with biologically favorable [estrogen-receptor-(ER) or progesterone-receptor-(PR) positive, HER2-negative] early-stage breast cancer participating in an Institutional Review Board (IRB) approved Phase I protocol (no. Pro00015617) at Duke University Medical Center were included in the study (trial registration no. NCT00944528). Patients (n ¼ 32) 55 years or older with cT1N0 invasive carcinomas (n ¼ 25) or low/intermediate grade ductal carcinoma in situ, 2 cm (n ¼ 7) were enrolled after providing informed consent. Formalin fixed and paraffin embedded (FFPE) tumor samples were obtained at the time of diagnosis. Patients were enrolled consecutively in cohorts of eight to receive a 15, 18 and 21 Gy dose to determine the maximum tolerated dose of preoperative partial breast radiotherapy. Surgical resection was performed on patients within 10 days of treatment and postirradiation FFPE tumor samples were obtained at the time of surgical excision.

### Microarray Analysis of Human Samples

Of 32 patients enrolled in the clinical trial, 26 had sufficient paired tumor tissue to be used for microarray analysis. In addition, we had a total of 9 biologic replicates to test for reproducibility of our data. RNA extraction and labeling was performed using the RNeasy FFPE kit (cat. no. 73504; QIAGENt, Valencia, Calif.), and the Sensation-Pluse FFPE Amplification and Labeling Kit (cat. no. 902312; Affymetrix Inc., Santa Clara, Calif.). All total RNA samples were assessed for quality using a NanoDrope 8000 spectrophotometer for absorbance ratios and the

Agilent Bioanalyzer 2100 (Agilent Technologies, Santa Clara, Calif.). Whole transcriptome expression analysis was evaluated with HTA 2.0 arrays (cat. no. 902162; Affymetrix). All samples were fully annotated and linked to clinical data.

The patients' genomic data discussed in this article has been deposited in the NCBI Gene Expression Omnibus (GEO) (http://www.ncbi.nlm.nih.gov/geo/) (13). The deposited data are accessible through the GEO Series accession no. GSE65505 (http://1.usa.gov/1JQF19x).

### Breast Cancer Cell Lines

A total of 16 breast cancer cell lines displaying gene expression patterns consistent with distinct clinical breast subtypes were selected (12, 14). Those cell lines were as follows: for luminal, MCF7, T47D, ZR751, CAMA-1, BT474 (Her2 [symbol] ), SKBR3 (Her2 [symbol] ), AU565 (Her2 [symbol] ); for basal, SUM149, SUM159, MDA-MB-231, MCF10A, MCF12A, BT549, HBL100, HCC1954 (Her2 [symbol] ) and DKAT. These cell lines reflect the heterogeneity of human breast cancer, thereby providing a valid biological model for study of phenotype-specific mechanisms. Cell lines were obtained from the American Type Culture Collection (ATCCt, Gaithersburg, Md.) with the exception of DKAT, ZR751, MCF10A and the SUM lines, which were a gift from Drs. Victoria Seewaldt and Gayathri Devi (15, 16). For full details of culture conditions, please see below.

### Microarray Analysis of Breast Cancer Cell Lines

Total RNA was collected from approximately 13106 cells in each of the 16 cell lines. RNA was isolated from the irradiated cell lines (5 Gy) 24 h after treatment using the RNeasy Mini Isolation Kit (QIAGEN). The same protocol was used for control cell lines (0 Gy). Samples were run in triplicate. RNA was hybridized to Affymetrix U133A2 arrays and processed in the Microarray Core Facility (Duke University Medical Center, Durham, N.C.).

The genomic data for the breast cancer cell lines discussed in this article have been deposited in the NCBI GEO (http://www.ncbi.nlm.nih.gov/geo/) (13). The deposited data are accessible though the GEO Series accession no. GSE59732 (http://1.usa.gov/1JQJF4j).

### Statistical Consideration

All statistical analyses were conducted using the R statistical environment and extension packages from CRAN and the Bioconductor project (17, 18). Gene level expression estimates were obtained with Affymetrix Expression Console software (v.1.3) using RMA-Sketch workflow. To check for sample outliers and batch effects, principal component analysis of the global gene expression was conducted. Batch effects were corrected by scale-shift normalization prior to analysis (19). For the human tumor samples, differential expression for paired samples was evaluated using the Bioconductor limma package with correction for multiple comparisons (20). Genes with false discovery rate (FDR) adjusted P values (Q values) less than 0.05 were selected as differentially expressed in response to radiation exposure (21). In the breast cancer cell lines, predefined genes of interest were selected to represent major cellular signaling pathways of interest in cancer progression and radiation response. A two-sample t test, followed by multiple hypotheses testing, was performed to identify differentially expressed probe sets caused by radiation exposure. To link the human data to our tissue culture findings, we utilized a two-way multiplicative linear mixed-effects model (22) (using the cell line as a random effect) to identify interactions between the 100 human genes whose expression was most significantly changed by exposure to radiation and differentially expressed genes in the radiation-responsive versus nonresponsive breast cancer cell lines. Implementation was provided by the linear mixed effects (lme) function in the nonlinear mixed effects (nlme) extension package and the inference was conducted using the restricted maximum likelihood (REML) approach (23). The null sampling distribution for testing the marginal hypothesis of each fixed effect was assumed to follow a t distribution with appropriate degrees of freedom. The analyses were conducted under the implicit assumption of homogeneity with respect to the variance of the random effects and the variance of the measurement errors. Multiplicity was addressed within the FDR framework as above. The interaction effect between hypothesized radiation response status and radiation treatment is considered significant for those probe sets with FDR adjusted P values (Q values) less than 0.05.

### The Cancer Genome Atlas (TCGA)

We used TCGA for external confirmation of our results. TCGA mRNA data were retrieved from the Cancer Genomic Data Server (CGDS) through the Computational Biology Center Portal (cBio; http://www.cbioportal.org/). The cdgsr extension package (https://cran.r-project.org/web/packages/cgdsr/cgdsr.pdf) was used to execute the retrieval. We used a validated radiation response molecular signature (AR, cJun, STAT1, PKC, Re1A, cABL, SUMO1, CDK1, HDAC1 and IRF1) (24) to calculate the radiosensitivity index (RSI) for 81 basal-like TCGA breast tumor samples. The model predicts a continuous RSI that is based on the survival fraction at 2 Gy (SF2). We did a linear regression of FAS expression values over RSI for all basal samples.

### Immunohistochemistry (IHC)

FFPE tissue samples were processed via standard histologic procedures and 5 lm sections were stained with the intelliPATHt automated immunostainer (Biocare Medical Inc., Concord, Calif.). After an antigen retrieval step (35 min in sub-boiling citrate buffer, pH 6.0), the anti-FAS primary antibody [Ab-FAS (C20); no. sc715; Santa Cruz Biotechnology Inc., Dallas, Tex.] was applied for 2 h (1:800 dilution). The MDA-MB-468 cell line and placental tissue were used as positive controls. A negative control stain without primary antibody was also performed for all experiments. Each section was scored for average staining intensity ( [symbol] ¼ weak, 2 [symbol] ¼ moderate; there was no strong staining) and percentage of positive cells in both the cell membrane and the cytoplasm by a breast pathologist (JG). Those two factors were then multiplied together to create a modified histoscore. The membrane and cytoplasmic scores were added to generate a combined score. If the difference in combined score was 0.100, the expression level was considered significantly altered.

### Quantitative Real-Time RT-PCR Analysis

Total RNA was harvested from approximately 1×10⁶ irradiated cell lines (0 and 5 Gy) 24 h after irradiaton using the RNeasy Mini Isolation Kit (QIAGEN). RNA was reverse transcribed with MuLV reverse transcriptase and Oligo d(T) primers (Applied Biosystems, Foster City, Calif.). The SYBRt Green PCR Kit (Applied Biosystems) was used for quantitative real-time RT-PCR analysis. The primers were synthesized by Integrated DNA Technologies (Coralville, Iowa). Human primer sequences are listed as below. Relative differences in gene expression among groups were determined from cycle time (Ct) values. These values were first normalized to 18S or GAPDH in the same sample (ΔCt) and expressed as fold over control (2-ddC t). Realtime fluorescence detection was performed using an ABI PRISMt 7900 Sequence Detector (Applied Biosystems). The following primers were used: Forward: FAS-5′-TCA GTA CGG AGT TGG GGA AG-3′ (SEQ ID NO: 1) and FAS L-5′-GCA CTT TGG GAT TCT TTC CA-3′ (SEQ ID NO: 2); reverse: FAS-5′-CAG GCC TTC CAA GTT CTG AG-3′ (SEQ ID NO: 3) and FAS L-5′-CCT CCA TTT GTC TGG CTC AT-3′ (SEQ ID NO: 4).

### Western Blot Analyses

Protein expression levels were analyzed using Western blot assays. Cell lysates were prepared from cells at 50-70% confluence. Cells were harvested by trypsinization, washed once in PBS and resuspended in mammalian cell lysis buffer (MCLB) [50 mM Tris-HCl (pH 8.0), 100 mM NaCl, 5 mM EDTA, 2 mM DTT, 1% NP40, 10 mM b-glycerophosphate, 1 mM Na3VO4] supplemented with 13 protease inhibitor cocktail (cat. no. P8340; Sigma-Aldrich LLC, St. Louis, Mo.). After clarifying the extract by centrifugation, protein concentration was determined using the Bio-Rad DC protein assay (Bio-Rad Laboratories, Inc., Hercules, Calif.). Samples containing equal amounts of protein were mixed with equal volumes of 23 Laemmli sample buffer [125 mM Tris-HCl (pH 6.8), 4% SDS, 20% glycerol] containing 5% b-mercaptoethanol, boiled and separated by SDSPAGE. Proteins were transferred to nitrocellulose and probed with antibodies against: FAS (cat. no. 8023; Cell Signaling Technology, Danvers, Mass.), FAP1 (cat. no. sc-15356; Santa Cruz Biotechnology), TP53 (cat. no. MS-187-PO; Lab Vision/NeoMarkers, Fremont, Calif.), cleaved (active) caspase 3 (Asp175, cat. no. 9661S; Cell Signaling Technology), cleaved PARP [c-PARP (Asp214, cat. no. 9541, Cell Signaling Technology], cleaved caspase 8 (cat. no. NBP1-71399; Novus Biologicals LLC, Littleton, Colo.) and cleaved caspase 9 antibody (cat. no. AB3629; EMD Millipore, Billerica, Mass.).

### Modulation of FAS Expression

shRNA-Mediated FAS Knockdown.

To evalate the effect of FAS knockdown on radiosensitivity we used the radiation-responsive MCF7 cell line, which showed high levels of FAS induction in response to radiation exposure. We utilized a lentivirus-based approach to knockdown our gene of interest (cat. no. 9606102-9606106; Thermo Scientific Open Bio Systems, Huntsville, Ala.). The shRNA lentiviral vectors contained a U6 RNA polymerase III constitutive promoter that drives short hairpin RNA expression. The MCF7 cells were infected with a single human FAS shRNA (cat. no. 9606104; Thermo Scientific Open Bio Systems) or negative control shRNA lentiviral vectors and 72 h post infection were placed under puromycin (1 lg/ml) selection. A pool of five human FAS shRNAs (cat. no. 9606102-9606106) was also utilized in a separate experiment. Cells were maintained for 3-5 days in selection media. Roughly 70% of the cells survived. The surviving cells were considered to be stably expressing shRNA due to the high efficiency integration of lentiviral vector into the mammalian genome and puromycin resistance. Changes in FAS protein expression were confirmed by Western blot.

### FAS Overexpression.

Overexpression of FAS was achieved using a lentivirus-based approach (cat. no. EX-G0198-Lv105; GeneCopoeiae, Rockville, Md.) in two basal cell lines with low levels of FAS expression, MDA-MB-231 and AU565. The lentiviral vector contains a CMV promoter that derives human FAS expression. The cells were infected with FAS-vector or negative control lentiviral vectors and 72 h post infection the cells were placed under puromycin selection (1 mg/ml). Cells were selected further for another 3-5 days, and roughly 70% of cells survived. The surviving cells were considered to be stably expressing FAS due to high efficiency of lentiviral vector integration into the mammalian genome. Change in FAS protein expression was confirmed by Western blot. FAS overexpres sing cells were then treated with radiation and a FAS agonistic antibody (CH11, cat. no. 05-201; EMD Millipore) to evaluate downstream FAS signaling.

### FAS Stimulating Antibody

To activate FAS, we used a FAS agonistic (stimulating) antibody, CH11 (monoclonal mouse antibody, cat. no. 05-201; EMD Millipore), at a concentration of 0.1 lg/ml. The goal was to determine if FAS activity would change radiation sensitivity in a radiation-nonresponsive cell line. SUM159 was pretreated with CH11 for 6 h prior to irradiation. The antibody-containing media was then removed and replaced with fresh media without antibody. Clonogenic cell survival assays were conducted as described below. Cell survival in the treated lines was compared to the untreated cell lines.

### Clonogenic Survival Assays

Cell lines of interest were plated and allowed to adhere overnight at37° C. Plates were exposed to variable doses of radiation using a Cesium-137 Mark-I irradiator (J L Shepherd, San Fernando, Calif.). Media was removed and replaced 24 h after irradiation. The cells were allowed to grow into colonies over a period of 10-14 days or until the control plates grew visible colonies. The plates were then washed with 13 PBS, fixed with 10% methanol-10% acetic acid for 10 min and stained with a 0.4% solution of crystal violet for 10 min. A ColCounte colony counter (Oxford Optronix Ltd., Abingdon, UK) was used to image and count the number of colonies per plate using fixed sensitivity settings so that only colonies of 0.50 cells were counted. Plating efficiencies (PE) were calculated using the formula: PE ¼% number of colonies/number of cells seeded and normalized to the control/sham-irradiated plates. Surviving fraction (SF) was calculated using the formula: SF ¼ number of colonies/number of cells seeded3 PE. End points included the surviving fraction at each dose level.

shRNA-Mediated TP53 Knockdown

We utilized a retrovirus-based approach to knockdown TP53 in MCF7 cell lines (25) (gift from Dr. Xiaohu Tang). Briefly, the shRNA retroviral vector pRetroSuper-puro contained a H1 RNA polymerase III-constitutive promoter that drives short hairpin RNA expression. The MCF7 cells were infected with human TP53 shRNA or negative control shRNA retroviral vectors and 72 h after infection were placed under puromycin (1 lg/ml) selection. Cells were selected further for another 3-5 days, and roughly 70% of the cells survived. The surviving cells were considered to be stably expressing shRNA due to high-efficiency integration of the retroviral vector into the mammalian genome and puromycin resistance. Changes in TP53 protein expression were confirmed by Western blot.

### ImageStream Flow Cytometry

In addition to the effect of radiation on FAS expression at the mRNA and protein levels, exposure to radiation may also affect FAS localization in the cells. We used the ImageStreamXt Mark II imaging flow cytometer (Amnis/EMD Millipore, Seattle Wash.) to evaluate radiation-induced changes in the localization of: 1. FAS; 2. FAS associated phosphatase 1 (FAP1); and 3. FAS ligand. Human breast cancer cell lines (radiation-responsive cell line MCF7 and radiation non responsive cell line SUM159) were 0 or 5 Gy irradiated and harvested 24 h after irradiation. Cells were stained with mAbs for human FAS (FAS PE; eBioscience Inc., San Diego, Calif.) or FAS ligand (FASL, PE; BioLegendt Inc., San Diego, Calif.) After Ab staining the cells were fixed with 1% formaldehyde. For the cytoplasmic immunostaining, the cells were then permeabilized and blocked in permeabilization buffer (3% fetal calf serum, 0.1% Tritone X-100) at room temperature for 1 h and incubated with anti-FAS Ab, anti-FAS ligand Ab or anti-FAP1 Ab (H-300, Santa Cruz Biotechnology) followed by incubation with goat-anti-rabbit IgG FITC Ab (Santa Cruz Biotechnology) in permeabilization buffer. Anti-DRAQ5 DNA dye (Cell Signaling Technology) was used for nuclear staining. A total of 5,000-8,000 cells were then collected and analyzed by ImageStreamX Mark II imaging flow cytometer. Membrane and cytoplasmic masks were used to analyze FAS and FAS ligand localization. FAS and FAP1 co-localization was quantified by calculating the bright detail similarity of the FAS and FAP1 intensity.

### Cell Line Growth Conditions

Cell lines growth conditions: MCF7, ZR751, MDAMB231, and SUM159 were cultured in Dulbecco's modified Eagle's medium supplemented with 10% fetal bovine serum (Hyclone, SH30071.03) and 1× Antibiotic-Antimycotic (Gibco, 15240-062).

SUM149 cells were grown in Ham's F-12 medium supplemented with 5% fetal bovine serum (Hyclone, SH30071.03), 5 ug/ml Insulin (Sigma-Aldrich®, I-5500), 1 ug/ml Hydrocortisone (Sigma-Aldrich®, H-4001) and 1× Antibiotics/Antimycotic (Gibco, 15240-062).

T47D cells were grown in MEM-alpha medium (Gibco, 12561) supplemented with 6% of fetal bovine serum (Hyclone, SH30071.03), 12 mM Hepes (Gibco15630), 1× MEM NEAA(Gibco 11140), 1 mM Sodium Pyruvate (Gibco, 11360), 1 ug/ml insulin (Sigma-Aldrich®, 1-5500), 1 ug/ml Hydrocortisone (Sigma-Aldrich®, H-4001), 12.5 ng/ml EGF (Sigma-Aldrich®, E4127) and 1× Antibiotic-Antimycotic (Gibco, 15240-062).

AU565 and HCC1965 cells were grown in RPMI1640 medium supplemented with 10% of fetal bovine serum (Hyclone, SH30071.03), 1× Sodium Pyruvate (Gibco 11360-070), 10 mM Hepes (Gibco, 15630) and 1× Antibiotic-Antimycotic (Gibco, 15240-062).

BT474 and SKBR3 cells were grown in RPMI1640 medium supplemented with 10% of fetal bovine serum (Hyclone, SH30071.03) and 1× Antibiotic-Antimycotic(Gibco, 15240-062).

BT549 were grown in RPMI1640 medium supplemented with 10% of fetal bovine serum (Hyclone, SH30071.03), 1× Sodium Pyruvate (Gibco 11360-070), 10 mM Hepes(Gibco, 15630), 8 ug/ml Insulin (life technology 12585014) and 1× Antibiotic-Antimycotic(Gibco, 15240-062).

CAMA1 cells were grown in MEM (Gibco 11095-080) medium supplemented with 10% of fetal bovine serum (Hyclone, SH30071.03), 1× Sodium Pyruvate (Gibco 11360-070), 1× NEAA (Gibco 11140) and 1× Antibiotic-Antimycotic (Gibco, 15240-062).

HBL100 cells were grown in McCoy's 5a medium (Gibco 16600-082) supplemented with 10% of fetal bovine serum (Hyclone, SH30071.03) and 1× Antibiotic-Antimycotic (Gibco, 15240-062).

MCF10A cells were grown in DMEM/F12 (GIBCO 11330-032) medium supplemented with 5% of Horse Serum (Invitrogen #16050-122), 0.5 ug/ml of hydrocortisone(Sigma-Aldrich®, H4001), 10 ug/ml of Insulin (life technology 12585014), 20 ng/ml of EGF(Sigma-Aldrich®, e9644) and 1× Antibiotic-Antimycotic (Gibco, 15240-062).

MCF12A cells were grown in DMEM/F12 (GIBCO11330-032) medium supplemented with 10% of fetal bovine serum (Hyclone, SH30071.03), 1 ug/ml of hydrocortisone (Sigma-Aldrich, H-4001), 5 ug/ml of Insulin (life technology 12585014) and 1× Antibiotic-Antimycotic (Gibco, 15240-062).

DKAT cells were grown in MEGM (Lonza, CC-3150) medium and 1× Antibiotic-Antimycotic (Gibco, 15240-062).

All cell lines were maintained in a humidified incubator with 5% CO₂ atmosphere at 37° C.

### Results

### Gene Expression Response to Radiation in Human Breast Tumors

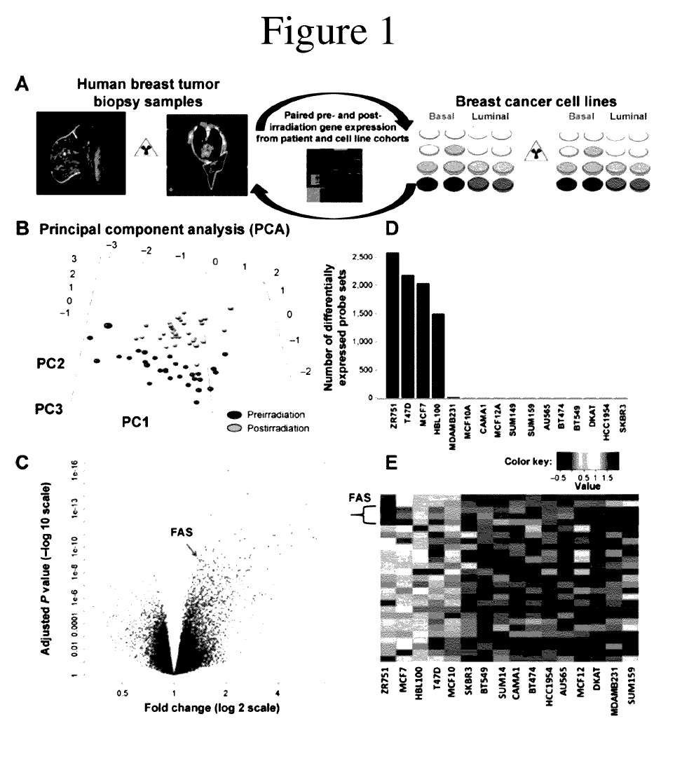

Thirty-two women with early-stage ER-positive breast cancer were enrolled in our novel clinical trial evaluating preoperative radiation therapy (FIG. 1A). After pathologic confirmation of a ≦2 cm in situ or invasive breast cancer with clinically negative lymph nodes, patients received a single fraction dose of 15, 18 and 21 Gy. Within 10 days, patients were taken to the operating room for surgical excision. Twenty-six patients had tumor tissue from the pretreatment diagnostic core biopsy and post-treatment from the surgical specimen collected for further analysis of gene expression.

Radiotherapy to the intact breast tumor resulted in marked changes in gene expression (FIG. 1B). Principal component analysis showed distinct separation between pre- and postirradiation breast tumor samples with minimal overlap. Furthermore, gene induction, rather than repression, appears to be the dominant effect, with a large number of genes induced by radiation exposure (FIG. 1C). From this data, 100 genes with the most significant change in radiation response (Q less than 2.57 E-08) were selected for further evaluation (FIG. 1C, green dots). Genes in this list included those involved in various biological processes such as apoptosis, cell cycle and MAPK signaling pathways.

### Gene Expression Response to Radiation in Breast Cancer Cell Lines

Our clinical cohort consisted entirely of patients with luminal subtype breast cancer, since individuals with more aggressive subtypes were not felt to be optimal candidates for the clinical trial. To better understand the biologic relevance of our findings across tumor subtypes, we next evaluated gene expression patterns after irradiation in a large panel of biologically diverse breast cancer cell lines (FIG. 1A). Microarray analysis indicated markedly different radiation response profiles among the breast cancer cell lines after irradiation, with two dominant patterns emerging (FIG. 1D). In four of the 16 cell lines, there were a large number of genes exhibiting significant change in expression as a result of radiation exposure. Three of the four HER2-luminal cell lines (MCF7, T47D, ZR751) and one basal line (HBL100) were included in this radiation-responsive cohort. In contrast, using the same filtering criteria, we noted a striking absence of change in gene expression in the remaining 12 basal and HER2 [symbol] luminal cell lines.

Candidate genes were evaluated to determine which specific pathways might be driving the unique radiation response in these two cohorts. The most significant differential pattern of expression was seen in the induction of FAS after irradiation. FAS (CD95, APO-1) a critical modulator of programed cell death, was seen to increase significantly in three of the four radiation-responsive cell lines (MCF7, ZR751 and HBL100). In contrast, despite highly variable levels of FAS expression at baseline, none of the radiation-nonresponsive cell lines demonstrated significant FAS induction. This effect was conserved across multiple FAS probe sets. Additional genes important in programed cell death, TP5313 (p53-inducible protein 3) and caspase 9, were also noted to be differentially affected by radiation in the radiation-responsive versus nonresponsive cell lines.

### Concordant Gene Expression Response in Breast Tumors and Cell Lines

To link the human tumor samples and biologically diverse breast cancer cell lines, we assessed for differential expression of the 100 most radiation-responsive human genes in the radiation-responsive versus nonresponsive cell lines. Fourteen genes associated with 27 probe sets were identified as having significant Q values for differential expression in both the human data and in the cell line data (Q, 0.002). FAS was again identified as highly significant (Q ¼ 7.42E-10) (FIG. 1E). These findings suggested a role for the differential activation of the apoptosis pathway as a biologically plausible explanation for clinical differences in radiation response (10) that deserved further exploration.

### Validation of FAS Induction in Human Tissue

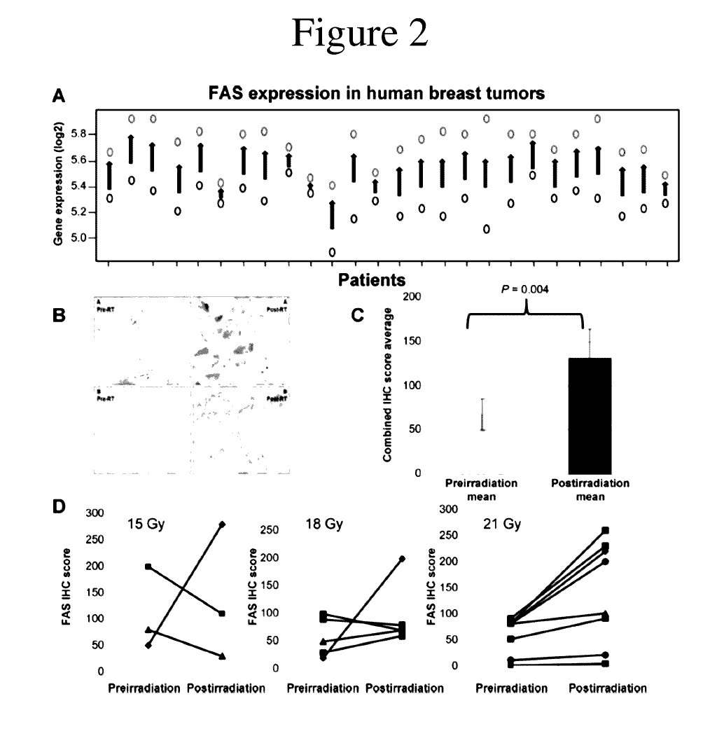

To validate our human microarray results (FIG. 2A) showing that in each patient FAS was induced in response to radiation exposure, we utilized immunohistochemistry (IHC) to confirm FAS induction at the protein level. We again saw significant FAS induction with radiation exposure (FIG. 2B). For evaluable preirradiation patient samples (n ¼ 27), the mean score was 68, while evaluable postirradiation samples (n ¼ 20) had a mean FAS score that was nearly double at 132 (P ¼ 0.004; FIG. 2C). Sixteen patients had paired pre- and postirradiation IHC results. Six of those (38%) demonstrated upregulation of FAS that was identified as significant (change in histoscore 0.100; P 1/40 0.01). Although induction was not as uniform at the protein level, tumors were assessed at a variable time window (1-10 days) between radiation treatment and surgical resection in this initial proof-of-concept dose-escalation trial, and IHC is known to be a more subjective measure. Of the six cases with significant induction, four were in the highest radiation dose cohort of 21 Gy suggesting that significant FAS induction may be dose dependent (FIG. 2D).

### FAS Validation in Cell Lines

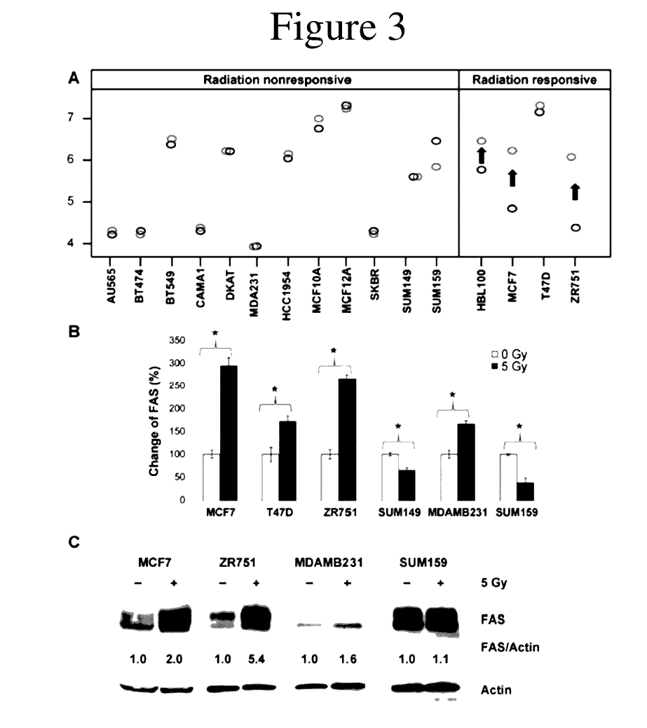

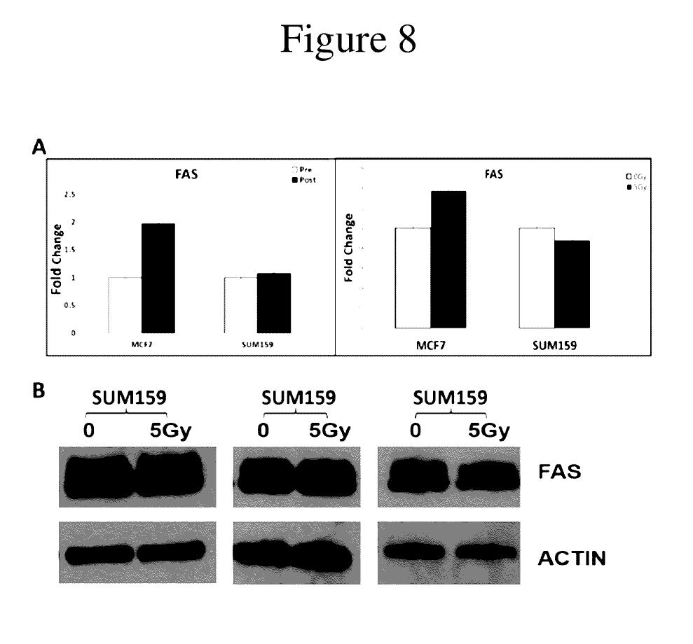

To confirm our cell line microarray findings (FIG. 3A) showing induction of FAS in predominantly luminal cell lines, we selected representative subsets of cell lines from the radiation-responsive and nonresponsive groups for further study. Although FAS induction was seen in one of the nonresponsive cell lines (MDAMB231), qPCR analysis also confirmed significant induction of FAS in three of the radiation-responsive lines (MCF7, ZR751 and T47D). In contrast, the two SUM lines did not demonstrate FAS induction. Similarly, at the protein level, Western blot analysis demonstrated that the radiation-responsive cell lines (MCF7 and ZR751) had increased FAS signal in response to radiation while there were no significant changes in MDAMB231 and SUM159 (radiation-nonresponsive subset) despite significant variability in baseline FAS levels (FIG. 3C). Our findings of FAS induction in the radiation-responsive cell line MCF7 and no change or repression in the nonresponsive line SUM159 were confirmed over multiple experiments [FIG. 3 and FIG. 8].

### FAS Localization

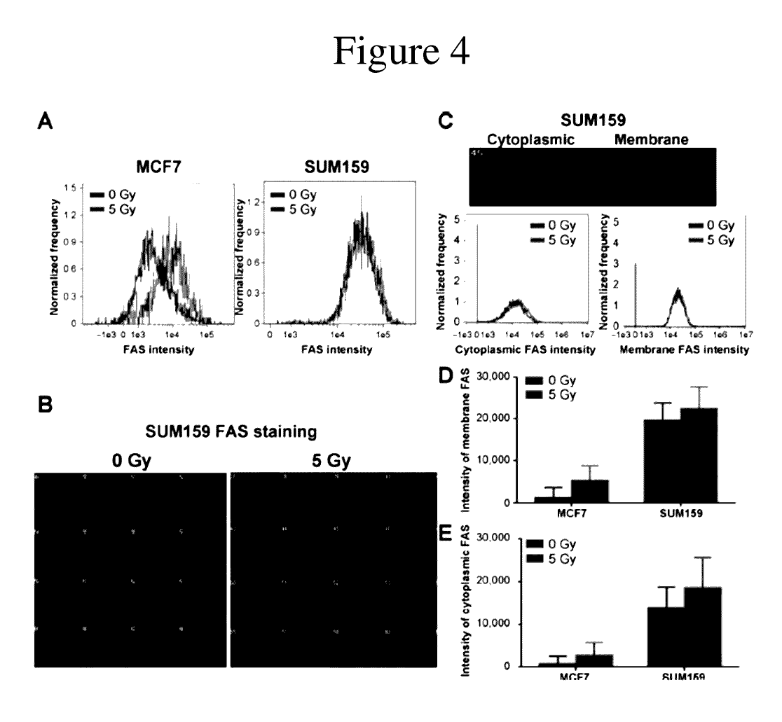

It has been shown that in some human tumors, export of FAS to the cell surface is impaired due to FAS association with FAS-associated phosphatase 1 (FAP1), thereby inactivating FAS ligand-mediated apoptosis (29, 30). To investigate if radiation exposure affected FAP1 expression and FAS retention differently in radiation-responsive versus nonresponsive cell lines, we used ImageStream flow cytometry to analyze FAS trafficking and FAS/FAP1 colocalization. We found that radiation exposure increased total FAS intensity in the radiation-responsive cell line MCF7, but had little effect on the radiation-nonresponsive cell line, SUM159 (FIG. 4A). In SUM159 cells, FAS was intensely expressed on both the cell surface and in the cytoplasm before as well as after exposure to radiation (FIG. 4B-E).

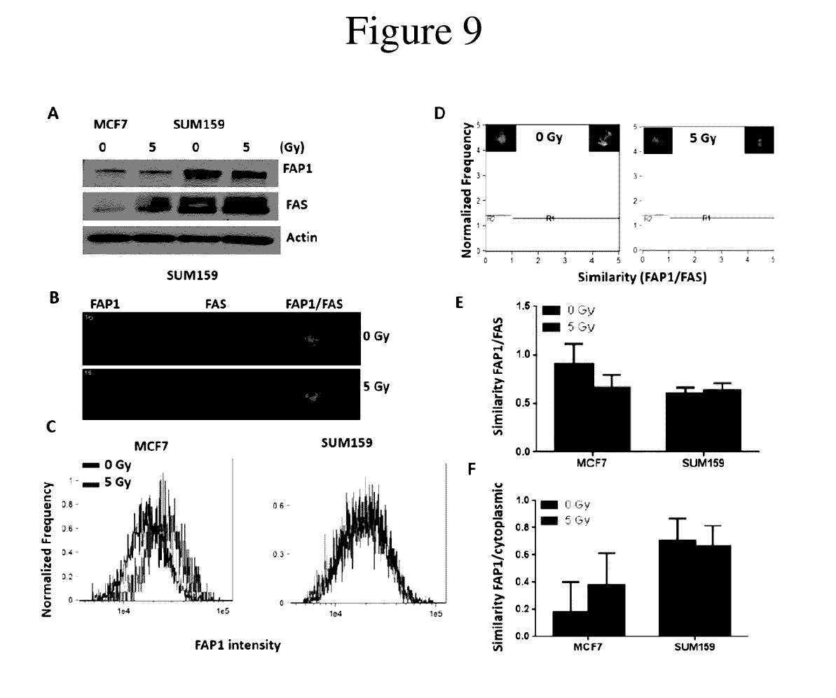

FAP1 was also highly expressed in the cytoplasm of the radiation nonresponsive cell line SUM159. Radiation exposure increased FAP1 expression in MCF7, but not in SUM159 (FIG. 9A-C). In both cell lines, the similarity scores of FAP1 and total FAS or FAP1 and cytoplasmic FAS were low (less than 1) despite radiation exposure, indicating there was low co-localization of FAP1 and FAS (FIG. 9D-F) and as a result, that FAS was not retained in the cytoplasm through association with FAP-1.

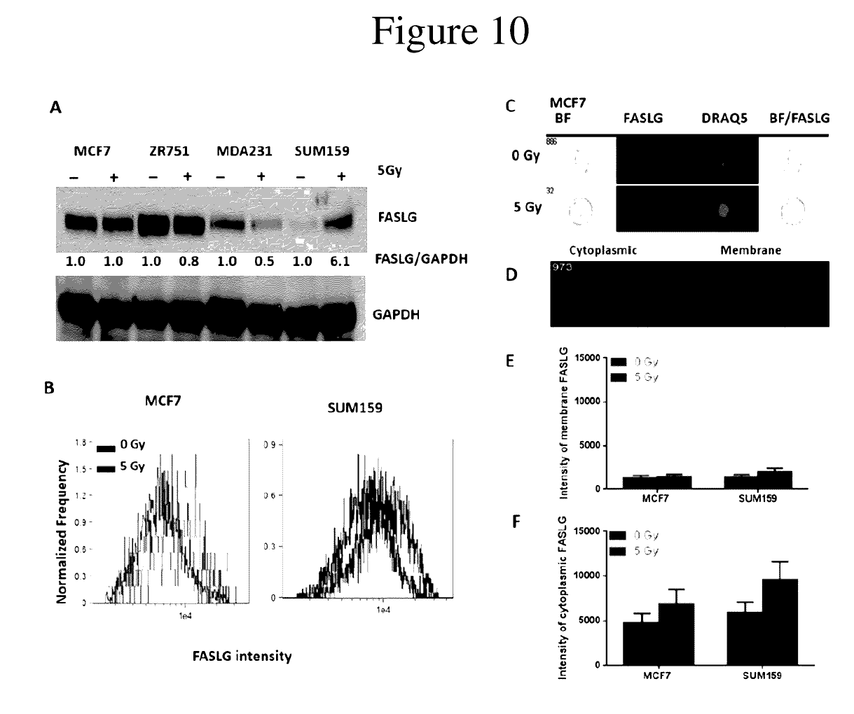

Finally, some increase in total FAS ligand expression was noted after irradiation in both cell lines (FIG. 10A-D). However FAS ligand was contained primarily in the cytoplasm with minimal surface expression (FIGS. 10E and F) both before and after irradiation. SUM159 did exhibit a small increase in surface expression after irradiation but no clear relationship could be discerned between FAS ligand localization and patterns of FAS expression. Similarly, no easily discernible pattern could be identified in FAS ligand mRNA expression and induction or repression of FAS (data not shown) across all 16 cell lines.

### TP53 and FAS Induction

Next, we investigated whether the induction of FAS after irradiation was driven by TP53, which is a known key mediator of cell cycle control, programed cell death and radiation response, as well as an upstream regulator of FAS. Although TP53 is commonly mutated in breast cancers overall, only 12% of luminal A tumors contain TP53 mutations compared to 80% of basal-like tumors.

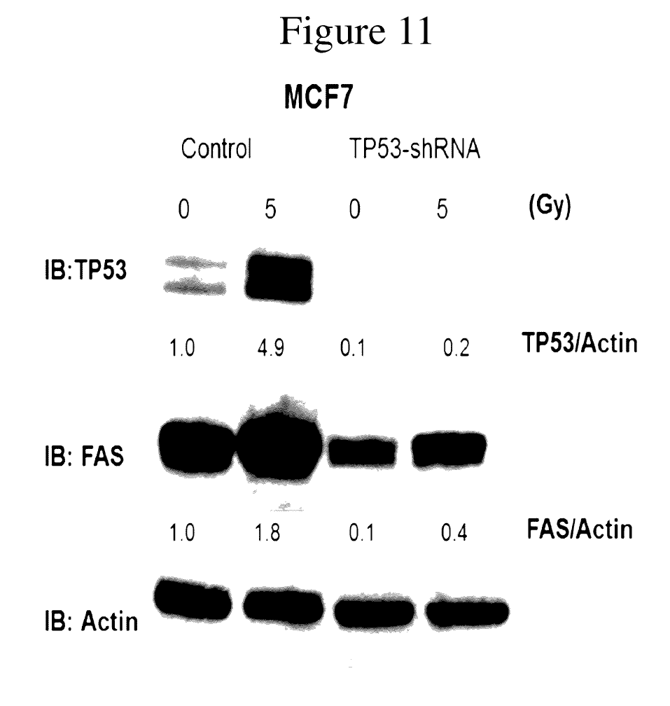

The MCF7 cell line is known to express wild-type TP53 and induce both TP53 and FAS in response to radiation exposure (FIG. 11). Therefore, we used shRNA to knockout TP53 in the MCF7 cell line. However, despite the loss of TP53, FAS induction was persistent in the TP53 null cell line. The overall level of FAS expression was reduced, but the magnitude of radiation response was similar to that seen in the parent MCF7 cell line [FAS expression (normalized to actin): 1.0-1.8 after irradiation in TP53 wt; 0.1-0.4 in TP53 null]. It is important to note that in addition to TP53 there are other known transcriptional regulators that can affect FAS expression, including nuclear factor of kappa light polypeptide gene enhancer in B cells 1 (Nfkb1 or NF-kappaB), signal transducer and activator of transcription 3 (STAT3) and the jun proto-oncogene (JUN, also known as c-Jun) (31-33). Therefore, although FAS radiation response occurs in the absence of TP53 in our study, further study will be required to determine the role of additional regulators of FAS transcription.

### Impact of FAS Modulation on Radiation Sensitivity

Although FAS induction appeared to be differentially induced in the more favorable, luminal-type breast cancers, it was not clear that this could then be utilized as a tool to modify radiation response. Therefore, we selected one cell line from the responsive cohort (luminal: MCF7) and one from the nonresponsive cohort (basal: SUM159) to determine if sensitivity to radiation would change as a result of FAS modulation.

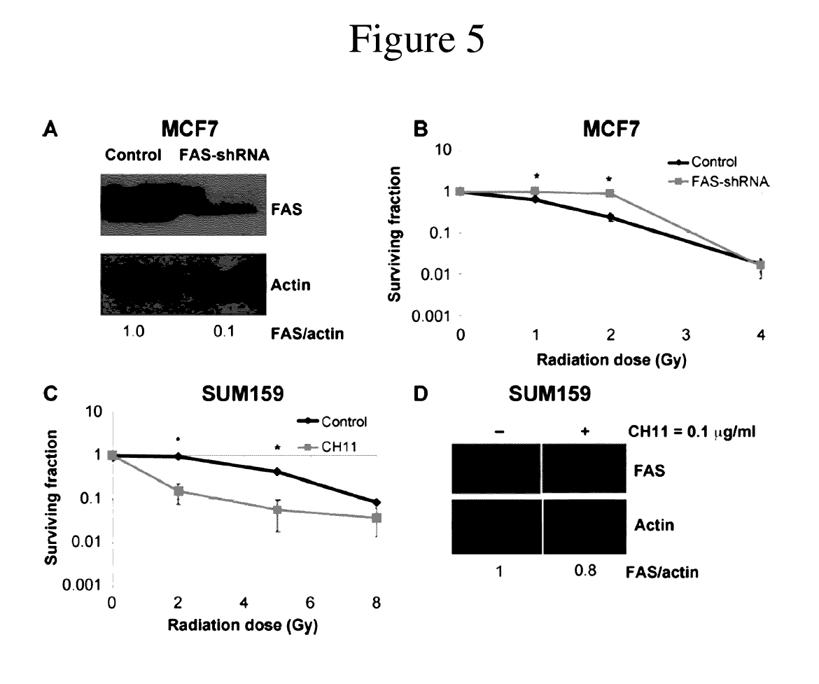

We selected the radiation-responsive MCF7 cell line to determine if radiation sensitivity could be reversed in the absence of FAS expression. We used Western blotting to first verify that shRNA effectively knocked down the FAS receptor (FIG. 5A). Radiation was also applied and no FAS induction was seen in the knockdown. Clonogenic cell survival assays were then performed and compared to the control shRNA MCF7 cell line with intact FAS (FIG. 5B). Both cell lines were equally radiosensitive at 4 Gy, but increased radioresistance was observed with FAS knockdown for the lower doses of 1 and 2 Gy.

In contrast, when FAS was activated by FAS-stimulating antibody CH11, the radiation-nonresponsive SUM159 demonstrated significantly greater radiosensitivity (FIG. 5C). The dose to achieve a surviving fraction of 0.1 was approximately 8 Gy in the control cells and 3.25 Gy in the CH11-treated SUM159 cells, yielding a dose-modifying factor of 2.5. This occurred despite a lack of visible FAS induction (FIG. 5D).

### FAS as a Therapeutic Target in Basal Cancers

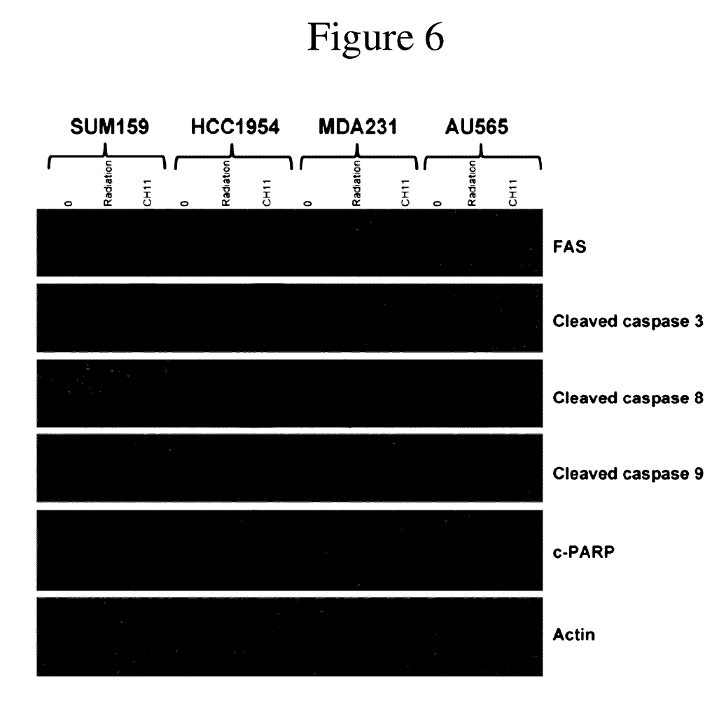

Based on our findings that SUM159 could be sensitized to radiation using a FAS-activating antibody (despite a lack of FAS induction), we hypothesized that basal cell lines with high levels of FAS may retain an intact apoptosis signaling pathway while those with low-level expression do not. Western blot analysis revealed that in the cell lines with high baseline levels of FAS (SUM159, HCC1954) downstream activation of cleaved caspases 3, 9 and PARP did not occur in response to radiation, but could be induced with FAS stimulation via CH11. In contrast, those cell lines with low levels of FAS (MDA231, AU565) at baseline did not exhibit evidence of apoptosis after irradiation or stimulation with CH11 (FIG. 6).

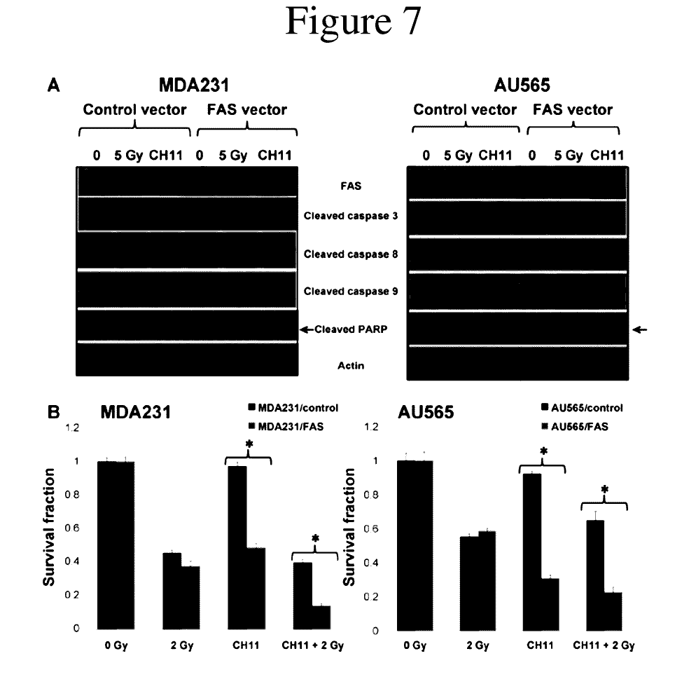

FAS was then overexpressed in the two cell lines with low baseline FAS expression, MDA231 and AU565, to determine if this could restore FAS function and as a result, radiation response. Western blot analysis revealed that significant induction of apoptosis proteases caspases 3, 8 and 9 and c-PARP could be seen in response to treatment with CH11 after FAS overexpression (FIG. 7A). Furthermore, reintroduction of FAS signaling was able to enhance radiation response (FIG. 7B) in both basal cell lines.

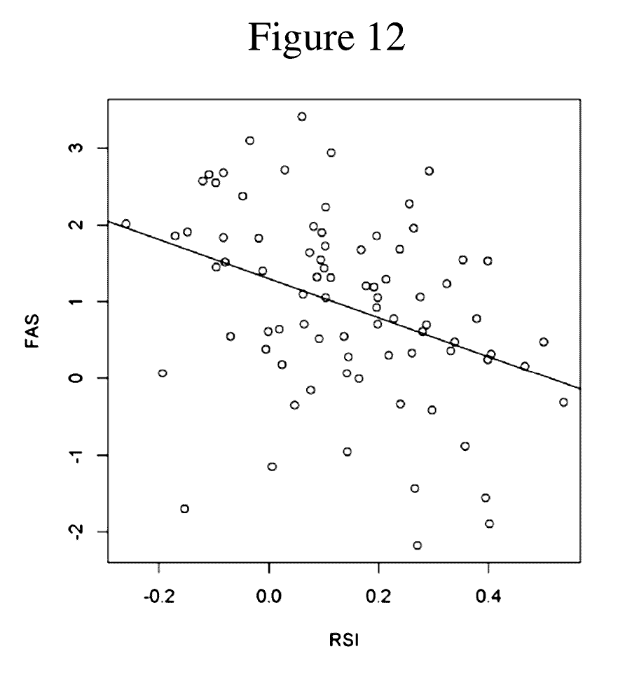

Finally, to further test our theory that baseline FAS expression may be linked to radiation sensitivity in the basal subtype, we utilized the validated radiation response molecular signature from Eschrich et al. (24) to calculate the radiosensitivity index (RSI) for 81 TCGA basal breast tumor samples. A linear regression model of FAS expression values over RSI demonstrated significance for all of the samples (P′%0.002844) with a negative regression coefficient (_0.83) meaning that FAS expression decreased as RSI increased; an increasing RSI score indicates radiation resistance (FIG. 12).

### Discussion

Delivery of radiation therapy in the treatment of breast cancer is currently based on istoric data that predated modern molecular techniques and an understanding of breast subtype-specific biology. For almost 300,000 women in the U.S. diagnosed with breast cancer each year, the “one size fits all” radiotherapy approach is a routine part of multidisciplinary breast cancer care. However, clinical data are accumulating to support the idea that radiation response can be linked to breast cancer phenotype (6, 7). A recent Early Breast Cancer Trialists' Collaborative Group (EBCTCG) publication, with pooled data on over 10,000 breast cancer patients from 17 historical randomized clinical trials, found that while radiation therapy was effective in both groups, the degree of risk reduction for any first recurrence was substantially greater in the estrogen receptor (ER [symbol] ) patients receiving radiation than those with ER-tumors (10). This data suggest that there is some biologic feature common to ER [symbol] tumors that enhances their sensitivity to ionizing radiation (10). If this characteristic can be identified, it can lead to a greater understanding of breast cancer radiation response and potentially influence radiotherapeutic approaches for all breast tumor subtypes.

Individualization of therapy is particularly relevant in breast cancer. Systemic therapy has moved from a more uniform therapeutic approach to biologically based treatment over the last 5-10 years. Chemotherapy delivered prior to surgery has revolutionized our understanding of subtype-specific chemotherapy response and dramatically impacted clinical practice (34). While initial trials evaluating preoperative chemotherapy grouped all patients with breast cancer together, it has quickly become apparent that this is not an optimal approach. Modern trials now routinely separate patients from different chemotherapy response groups into distinct clinical trials with unique end points.

Therefore, it seems feasible that a better understanding of breast cancer subtype-specific radiation biology could similarly impact clinical practice. Radiation dose could potentially be decreased in patients with more radiosensitive tumors, thus decreasing radiation-related normal tissue damage. Conversely, supplemental therapies could be utilized in patients with more radioresistant tumors to reduce the unacceptably high rate of tumor recurrence with associated morbidity and mortality. However, molecular insight into this differential radiation response is needed to lay the groundwork for clinical trials evaluating individualized radiotherapy.

In this work, we set out to determine whether there were variations in radiation response, using RNA microarray data from a rare cohort of paired pre- and postirradiation breast tumor tissue and a library of 16 diverse breast tumor cell lines. We specifically noted the differential induction of FAS (also known as APO-1 or CD95), a member of the tumor necrosis factor receptor (TNF-R) family, in the luminal human samples and the four radiation-responsive cell lines, three of which were luminal and HER2−. FAS is a cell surface receptor that contains an intracellular “death domain” and plays a critical role in the initiation of apoptotic cell death. Differential expression of FAS has been implicated in tumor aggression, metastasis and resistance to both chemotherapy and radiation therapy (35-39). In breast cancer patients, Botti et al. noted significantly shorter disease-free and overall survival periods in 167 FAS-negative and FASL-positive stage I/II breast cancer patients (40). In contrast, higher expression levels of FAS are associated with smaller tumor size, negative lymph nodes and prolonged disease-free survival (41).

In addition to its prognostic role, FAS has also been shown to predict response to therapy. In human myeloma and T-cell leukemia cells, resistance to mitoxantrone and doxorubicin has been associated with reduced FAS expression. In tumors with epigenetically repressed FAS that are rescued, tumor growth can be suppressed and chemosensitivity restored (42). Furthermore, radiation sensitivity has been linked to FAS expression levels in patients with malignant lymphoma. In patients with rapid clinical tumor regression, FAS was identified at relatively low radiation doses. This was in contrast to the low or absent expression seen in patients with squamous cell tumors from the same anatomic region (43). While FAS induction is viewed as contributing to differences in radiation sensitivity among tumors of diverse origin, it may be that differential FAS induction also helps to explain the clinical differences in response to radiation that exist within a heterogeneous group of breast tumors.

In addition to showing that FAS is induced in response to radiation in a subset of biologically favorable breast cancer cell lines and patient tumors, we have demonstrated, more importantly, that we can alter radiation response by modulating FAS. In the initially radiation-responsive MCF7 cell line, we were able to show that reduction of FAS increased radiation resistance. Furthermore, we were able to demonstrate that the induction seen in response to radiation was reduced, but not dependent on TP53. This finding is supported by the work of Oh et al., who also identified FAS induction despite using multiple mouse models that were TP53 deficient (45). In parallel, we were also able to demonstrate that the radiation response of resistant cell lines (e.g., SUM159) with high baseline levels of FAS expression can be enhanced by a FAS-stimulating antibody. Importantly, this was seen even when FAS induction was not apparent, suggesting that baseline FAS levels may serve as a biomarker of radiation response in the basal tumor subtype. Further supporting this hypothesis, we were able to restore FAS signaling and enhance radiation response by overexpressing FAS in basal cell lines with low baseline expression.

However, our study does have limitations. For example, the classification of cell lines as radiation responsive versus radiation nonresponsive is a working model based on the number of genes differentially expressed in response to radiation exposure. Our analysis of radiation effect in these two cohorts may be biased since we are using the same dataset to classify cells into responsive versus nonresponsive. Furthermore, in our study, the relationship between breast cancer subtype and radiation response was not perfectly aligned. One basal cell line had significant response to radiation and one radiation-responsive luminal cell line did not have significant FAS induction. It is possible that some additional clinical or tumor feature is needed to determine radiation response. Indeed, in human breast cancers, although we observed FAS mRNA induction with radiation exposure in all samples, at the protein level we observed FAS induction in only a subset of samples. Furthermore, the patterns of FAS-dependent cell death in vivo may also differ significantly from what we have observed in vitro due to significantly higher levels of FAS ligand secretion from tumor-associated macrophages (26,27, 28). Additional in vivo work will be required to further delineate the impact of the tumor microenvironment on subtype-specific programed cell death. Most importantly, clinical proof that FAS can be used as biomarker to individualize breast radiotherapy will require robust clinical trials with long-term locoregional response end points.

Given the disparate rates of local control in breast tumor subtypes, we would anticipate that subtype-specific radiation knowledge would be utilized differently depending on the subtype. In radiation-responsive breast cancers with significant induction of FAS, clinical trials evaluating dosereduction strategies to limit the toxicity of therapy could be designed. In contrast, more nonresponsive tumors with high levels of FAS could be selected for concurrent targeted therapy. The use of clinical antibodies targeting the FAS receptor has thus far been limited by normal tissue toxicity (46), but alternative strategies are under investigation. One such strategy involves the use of short structured RNA molecules, known as aptamers, which have several advantages over antibody therapy including the potential to develop aptamer antidotes that reverse toxicity (47).

Our ongoing projects and future studies include using in vitro and in vivo assays to validate and further evaluate additional radiation response candidate genes, as well as to elucidate FAS interaction partners and their involved pathways in the radiation-responsive versus nonresponsive cell lines.

### CONCLUSION

We have linked FAS induction to luminal breast tumors, confirmed that this appears to be a subtype-specific phenomenon in our diverse cohort of breast cancer cell lines and demonstrated that FAS can be used to favorably affect radiation sensitivity in resistant subtypes. Based on this data we believe that FAS has the potential to act as a predictive and therapeutic biomarker for radiation response in breast cancer patients.

### REFERENCES FOR EXAMPLE 1

1. Perou C M, Sorlie T, Eisen M B, van de Rijn M, Jeffrey S S, Rees C A, et al. Molecular portraits of human breast tumours. Nature 2000; 406:747-52.
2. Carey L A, Dees E C, Sawyer L, Gatti L, Moore D T, Collichio F, et al. The triple negative paradox: primary tumor chemosensitivity of breast cancer subtypes. Clin Cancer Res 2007; 13:2329-34.
3. Rody A, Karn T, Solbach C, Gaetje R, Munnes M, Kissler S, et al. The erbB2 [symbol] ) cluster of the intrinsic gene set predicts tumor response of breast cancer patients receiving neoadjuvant chemotherapy with docetaxel, doxorubicin and cyclophosphamide within the GEPARTRIO trial. Breast 2007; 16:235-40.
4. Rouzier R, Perou C M, Symmans W F, Ibrahim N, Cristofanilli M, Anderson K, et al. Breast cancer molecular subtypes respond differently to preoperative chemotherapy. Clin Cancer Res 2005; 11:5678-85.
5. Kyndi M, Sorensen F B, Knudsen H, Overgaard M, Nielsen H M, Overgaard J. Estrogen receptor, progesterone receptor, HER-2, and response to postmastectomy radiotherapy in high-risk breast cancer: the Danish Breast Cancer Cooperative Group. J Clin Oncol 2008; 26:1419-26.
6. Nguyen P L, Taghian A G, Katz M S, Niemierko A, Abi Raad R F, Boon W L, et al. Breast cancer subtype approximated by estrogen receptor, progesterone receptor, and HER-2 is associated with local and distant recurrence after breast-conserving therapy. J Clin Oncol 2008; 26:2373-8.
7. Voduc K D, Cheang M C, Tyldesley S, Gelmon K, Nielsen T O, Kennecke H. Breast cancer subtypes and the risk of local and regional relapse. J Clin Oncol 2010; 28:1684-91.
8. Amundson S A, Do K T, Vinikoor L C, Lee R A, Koch-Paiz C A, Ahn J, et al. Integrating global gene expression and radiation survival parameters across the 60 cell lines of the National Cancer Institute Anticancer Drug Screen. Cancer Res 2008; 68:415-24.
9. Helland A, Johnsen H, Froyland C, Landmark H B, Saetersdal A B, Holmen M M, et al. Radiation-induced effects on gene expression: an in vivo study on breast cancer. Radiother Oncol 2006; 80:230-5.
10. Early Breast Cancer Trialists' Collaborative G, Darby S, McGale P, Correa C, Taylor C, Arriagada R, et al. Effect of radiotherapy after breast-conserving surgery on 10-year recurrence and 15-year breast cancer death: meta-analysis of individual patient data for 10,801 women in 17 randomised trials. Lancet 2011; 378:1707-16.
11. Palta M, Yoo S, Adamson J D, Prosnitz L R, Horton J K. Preoperative single fraction partial breast radiotherapy for earlystage breast cancer. Int J Radiat Oncol Biol Phys 2012; 82:37-42.
12. Neve R M, Chin K, Fridlyand J, Yeh J, Baehner F L, Fevr T, et al. A collection of breast cancer cell lines for the study of functionally distinct cancer subtypes. Cancer Cell 2006; 10:515-27.

13. Barrett T, Wilhite S E, Ledoux P, Evangelista C, Kim I F, Tomashevsky M, et al. NCBI GEO: archive for functional genomics data sets-update. Nucl Acids Res 2013; 41:D991-5.
14. Kao J, Salari K, Bocanegra M, Choi Y L, Girard L, Gandhi J, et al. Molecular profiling of breast cancer cell lines defines relevant tumor models and provides a resource for cancer gene discovery. PLoS One 2009; 4:e6146.
15. D'Amato N C, Ostrander J H, Bowie M L, Sistrunk C, Borowsky A, Cardiff R D, et al. Evidence for phenotypic plasticity in aggressive triple-negative breast cancer: human biology is recapitulated by a novel model system. PLoS One 2012; 7:e45684.
16. Evans M K, Tovmasyan A, Batinic-Haberle I, Devi G R. Mn porphyrin in combination with ascorbate acts as a pro-oxidant and mediates caspase-independent cancer cell death. Free Radic Biol Med 2014; 68:302-14.
17. Gentlemen R, Carey V, Huber W, Irizarry R, Duboit S. Bioinformatics and computational biology solutions using R and bioconductor. New York: Springer; 2005.
18. Gentleman R C, Carey V J, Bates D M, Bolstad B, Dettling M, Dudoit S, et al. Bioconductor: open software development for computational biology and bioinformatics. Genome Biol 2004; 5:R80.
19. Owzar K, Barry W T, Jung S H, Sohn I, George S L. Statistical challenges in preprocessing in microarray experiments in cancer. Clin Cancer Res 2008; 14:5959-66.
20. Gentleman R C, Carey V J, Bates D M, Bolstad B, Dettling M, Dudoit S, et al. Bioconductor: open software development for computational biology and bioinformatics. Genome Biol 2004; 5:R80.
21. Owzar K, Barry W T, Jung S H. Statistical considerations for analysis of microarray experiments. Cts-Clin Transl Sci 2011; 4:466-77.
22. Pinheiro J C, Bates D M. Mixed effects models in S and S-PLUS. Berlin Heidelberg New York: Springer; 2000.
23. Pinheiro J, Bates D, DebRoy S, Sarkar D, R Development Core Team. 2011. nlme: linear and nonlinear mixed effects models. R package version 3.1-102.2013. (http://CRAN.R-project.org/package¼nlme)
24. Eschrich S A, Fulp W J, Pawitan Y, Foekens J A, Smid M, Martens J W, et al. Validation of a radiosensitivity molecular signature in breast cancer. Clin Cancer Res 2012; 18:5134-43.
25. Brummelkamp T R, Bernards R, Agami R. A system for stable expression of short interfering RNAs in mammalian cells. Science 2002; 296:550-3.
26. Lewis C E, Pollard J W. Distinct role of macrophages in different tumor microenvironments. Cancer Res 2006; 66:605-12.
27. Gabrilovich D I, Ostrand-Rosenberg S, Bronte V. Coordinated regulation of myeloid cells by tumours. Nat Rev Immunol 2012; 12:253-68.
28. Kiener P A, Davis P M, Starling G C, Mehlin C, Klebanoff S J, Ledbetter J A, et al. Differential induction of apoptosis by fas-fas ligand interactions in human monocytes and macrophages. J Exp Med 1997; 185:1511-6.
29. Bennett M, Macdonald K, Chan S W, Luzio J P, Simari R, Weissberg P. Cell surface trafficking of Fas: a rapid mechanism of p53-mediated apoptosis. Science 1998; 282:290-3.
30. Ivanov V N, Lopez Bergami P, Maulit G, Sato T A, Sassoon D, Ronai Z. FAP-1 association with Fas (Apo-1) inhibits Fas expression on the cell surface. Mol Cell Biol 2003; 23:3623-35.
31. Wieckowski E, Atarashi Y, Stanson J, Sato T A, Whiteside T L. FAP-1-mediated activation of NF-kappa B induces resistance of head and neck cancer to Fas-induced apoptosis. J Cell Biochem 2007; 100:16-28.
32. Ivanov V N, Bhoumik A, Krasilnikov M, Raz R, Owen-Schaub L B, Levy D, et al. Cooperation between STAT3 and c-Jun Suppresses Fas Transcription. Mol Cell 2001; 7:517-28.
33. Ivanov V N, Krasilnikov M, Ronai Z. Regulation of Fas expression by STAT3 and c-Jun is mediated by phosphatidylinositol 3-kinase-AKT signaling. J Biol Chem 2002; 277:4932-44.
34. Rastogi P, Anderson S J, Bear H D, Geyer C E, Kahlenberg M S, Robidoux A, et al. Preoperative chemotherapy: updates of National Surgical Adjuvant Breast and Bowel Project Protocols B-18 and B-27. J Clin Oncol 2008; 26:778-85.
35. Jost P J, Grabow S, Gray D, McKenzie M D, Nachbur U, Huang D C, et al. XIAP discriminates between type I and type II FASinduced apoptosis. Nature 2009; 460:1035-9.
36. Landowski T H, GleasonGuzman M C, Dalton W S. Selection for drug resistance results in resistance to Fas-mediated apoptosis. Blood 1997; 89:1854-61.
37. Friesen C, Herr I, Krammer P H, Debatin K M. Involvement of the CD95 (APO-1/FAS) receptor/ligand system in drug-induced apoptosis in leukemia cells. Nat Med 1996; 2:574-7.
38. Kykalos S, Mathaiou S, Karayiannakis A J, Patsouras D, Lambropoulou M, Simopoulos C. Tissue expression of the proteins fas and fas ligand in colorectal cancer and liver metastases. J Gastrointest Cancer 2012; 43:224-8.
39. Toillon R A, Descamps S, Adriaenssens E, Ricort J M, Bernard D, Boilly B, et al. Normal breast epithelial cells induce apoptosis of breast cancer cells via Fas signaling. Exp Cell Res 2002; 275:31-43.
40. Botti C, Buglioni S, Benevolo M, Giannarelli D, Papaldo P, Cognetti F, et al. Altered expression of FAS system is related to adverse clinical outcome in stage I-II breast cancer patients treated with adjuvant anthracycline-based chemotherapy. Clin Cancer Res 2004; 10:1360-5.
41. Mottolese M, Buglioni S, Bracalenti C, Cardarelli M A, Ciabocco L, Giannarelli D, et al. Prognostic relevance of altered Fas (CD95)-system in human breast cancer. Int J Cancer 2000; 89:127-32.
42. Maecker H L, Yun Z, Maecker H T, Giaccia A J. Epigenetic changes in tumor Fas levels determine immune escape and response to therapy. Cancer Cell 2002; 2:139-48.
43. Ogawa Y, Nishioka A, Hamada N, Terashima M, Inomata T, Yoshida S, et al. Expression of fas (CD95/APO-1) antigen induced by radiation therapy for diffuse B-cell lymphoma: immunohistochemical study. Clin Cancer Res 1997; 3:2211-6.
44. Li Y, Kanki H, Hachiya T, Ohyama T, Irie S, Tang G, et al. Negative regulation of Fas-mediated apoptosis by FAP-1 in human cancer cells. Int J Cancer 2000; 87:473-9.
45. Oh D S, Cheang M C, Fan C, Perou C M. Radiation-induced gene signature predicts pathologic complete response to neoadjuvant chemotherapy in breast cancer patients. Radiat Res 2014; 181:193-207.
46. Gasparian M E, Chernyak B V, Dolgikh D A, Yagolovich A V, Popova E N, Sycheva A M, et al. Generation of new TRAIL mutants DR5-A and DR5-B with improved selectivity to death receptor 5. Apoptosis 2009; 14:778-87.
47. Rusconi C P, Roberts J D, Pitoc G A, Nimjee S M, White R R, Quick G, Jr., et al. Antidote-mediated control of an anticoagulant aptamer in vivo. Nat Biotechnol 2004; 22:1423-8.

### Example 2

### Preoperative Single-Fraction Partial Breast Radiotherapy—A Novel Phase I Dose-Escalation Protocol with Radiation Response Biomarkers

### Summary

This phase I dose-escalation trial evaluates the feasibility of single-dose preoperative partial breast irradiation delivered with external beam techniques in early stage breast cancer patients. No acute dose-limiting toxicity was observed at 15, 18, or 21 Gy. Paired pre- and post-radiation imaging and tumor biopsies offer unique insight into the biology of breast cancer radiation response.

### Purpose

Women with biologically favorable early stage breast cancer are increasingly treated with accelerated partial breast radiation (PBI). However, treatment-related morbidities have been linked to the large post-operative treatment volumes required for external beam PBI. Relative to external beam delivery, alternative PBI techniques require equipment that is not universally available. To address these issues, we designed a phase I trial utilizing widely available technology to 1) evaluate the safety of a single radiation treatment delivered preoperatively to the small-volume, intact breast tumor and 2) identify imaging and genomic markers of radiation response.

### Methods

Women 55 or older with clinically node negative, ER+ or PR+, HER2-, T1 invasive carcinomas or low-intermediate grade in situ disease ≦2 cm were enrolled (n=32). Intensity-modulated radiotherapy was used to deliver 15 Gy (n=8), 18 Gy (n=8), or 21 Gy (n=16) to the tumor with a 1.5 cm margin. Lumpectomy was performed within 10 days. Paired pre- and post-radiation MRI images and patient tumor samples were analyzed.

### Results

No dose-limiting toxicity was observed. At a median follow-up of 23 months, there have been no recurrences. Physician-rated cosmetic outcomes were good/excellent and chronic toxicities were grade 1-2 (fibrosis, hyperpigmentation) in patients receiving preoperative radiation only. Evidence of dose-dependent changes in vascular permeability, cell density, and expression of genes regulating immunity and cell death were seen in response to radiation.

### Conclusions

Preoperative single-dose radiotherapy to intact breast tumors is well-tolerated. Radiation response is marked by early indicators of cell death in this biologically favorable patient cohort. This study represents a first-step towards a novel PBI approach. Preoperative radiation should be tested in future clinical trials as it has the potential to challenge the current treatment paradigm and provide a path forward to identify radiation response biomarkers.

### Introduction

Partial breast irradiation (PBI) is increasingly utilized in the treatment of early stage breast cancer. As data supporting the efficacy and tolerability of PBI continue to accumulate, this conceptual approach has been incorporated into national radiation guidelines and deemed acceptable for use outside of a clinical trial in carefully selected patientsl. A number of techniques are available for treatment delivery, ranging from a single intra-operative treatment to one week of twice daily treatments. In fact, a phase III trial focused only on brachytherapy PBI delivery techniques completed enrollment in 2009 with 1300 patients (GEC-ESTRO; NCT00402519). In addition, early reports are emerging from two randomized trials (ELIOT, TARGIT) testing a single-fraction of radiation delivered in the operating suite directly to the tumor bed 2, 3. However, with the exception of external beam PBI, every technique requires specialized training or equipment that, relative to linear accelerator-based options, are not as widely available to all radiation oncology practitioners. As a result, in a recent interim analysis of the phase III NSABP B-39/RTOG 0413 partial breast trial, external beam PBI was utilized in 72.9% of patients randomized to the PBI arm 4.

However, suboptimal outcomes have been reported with post-operative external beam PBI. Hepel and colleagues reported high rates of grade 3-4 soft tissue fibrosis (8.3%) in 60 patients treated with external beam techniques 5. Jagsi and colleagues closed their study after only 34 patients secondary to high rates of unacceptable cosmesis after only 2.5 years 6. Both authors linked these adverse outcomes to the sizeable treatment volumes required to target a post-operative surgical seroma plus appropriate margin. Although other institutional series and phase II trials?-10, as well as the randomized NSABP B-39/RTOG 0413 trial 4 have reported low rates of long-term toxicity the Canadian phase III trial (RAPID) also noted adverse cosmetic outcomes (29% PBI versus 17% whole breast, p<0.001) at 3 years 11, suggesting that the results of external beam PBI could be improved upon.

To address these issues, we designed a novel phase I dose-escalation trial of single-dose preoperative radiation treatment in carefully selected favorable risk patients. This technique has numerous potential advantages: 1) it can be delivered with widely available radiation techniques; 2) the target volume is a small intact breast tumor and its adjacent tissue rather than a large postoperative seroma which significantly decreases the uninvolved breast tissue receiving high radiation doses; 3) more accurate targeting of the areas of subclinical disease surrounding the tumor may be possible, 4) smaller treatment volumes are amenable to dose escalation which can further accelerate treatment and improve accessibility for patients, and 5) the pre-operative approach provides a novel opportunity to study breast cancer radiation response. Our goal was to determine the feasibility, toxicity, and maximally tolerated dose (MTD) of this approach.

### Materials and Methods

### Eligibility Criteria

Patients 55 or older with cT1N0 invasive carcinomas or low/intermediate grade ductal carcinoma in situ ≦2cm were enrolled after providing informed consent, on our Institutional Review Board (Pro00015617) approved study. Patients were estrogen receptor (ER) positive and/or progesterone receptor (PR) positive, HER2− with no evidence of lymphovascular invasion (LVI) on diagnostic biopsy. Neoadjuvant therapy was prohibited.

### Pre-Treatment Imaging

A treatment planning breast magnetic resonance imaging (MRI) was performed in the Department of Radiation Oncology (n=21) or at a diagnostic MRI (n=11) facility. Most patients were scanned in the prone position (n=30) on a dedicated 4-Channel Breast Coil (GE Healthcare or Hologic (Bedford, Mass.)) with arms up. Two patients were scanned supine with a 4-Channel Torso Array Coil (GE Healthcare). T1 and T2-weighted imaging, and dynamic contrast enhanced (DCE) images were obtained on a scanner with at least 1.5 Tesla magnet strength for characterization of the target volume. Diffusion weighted images (DWI) were acquired when feasible. A planning CT scan (GE Light-speed RT, GE Medical System, Milwaukee, Wis.) was then conducted in the same position as the treatment planning MRI using a prone breast board (CDR systems Inc, Calgary, Alberta, Canada) or a BodyFix® (Elekta Oncology System, Stockholm, Sweden) supine board.

### Treatment Planning

The breast tumor, or gross tumor volume (GTV), and the titanium diagnostic biopsy clip (required for protocol entry) were identified on the treatment planning MRI in BrainLAB iPlan (BrainLAB, Heimstetten, Germany) or Eclipse (Varian Medical Systems, Palo Alto, Calif.) with the assistance of an expert breast radiologist (typically XX). T1 post-contrast MRI images were utilized to identify the area of enhancing tumor while T2 MRI images, as well as those without fat subtraction, helped to distinguish tumor from post-biopsy change. MR images were then fused with the CT planning images using manual rigid-body registration to align the biopsy marker and surrounding soft tissues.

A 1.5 cm uniform expansion margin was applied around the GTV to account for microscopic disease spread and create the clinical target volume (CTV). Data correlating tumor extent on MRI with pathologic disease extent was utilized to select the CTV width 12. The advantages of in situ targeting were felt to overcome the decrease in absolute volume of tissue treated with a 1.5 cm preoperative margin as compared to a 1.5-2 cm postoperative margin. The rationale for this has been described previously 13. An additional 0.3 cm margin was applied to allow for minor variation in patient positioning and generate the planning target volume (PTV). The first 5 mm of subcutaneous of tissue from the external body surface was excluded from the CTV and PTV. The skin was defined in this protocol as a 3 mm layer from the external body surface. Breasts, heart, and lungs were segmented according to institutional standard and reflecting the guidelines of NSABP B39/RTOG 0413.

Four to seven beam co-planar or non-co-planar intensity-modulated therapy (IMRT) techniques were used to generate the radiation plan. Dose was prescribed to cover at least 95% of the CTV. Normal tissue constraints were developed using the NSABP/RTOG PBI trial and available hypofractionation literature 14 as a guide [Supplemental Table 1]. Additional treatment planning and delivery details are reported in a separate manuscript 15.

### Treatment

Patient positioning was confirmed with on-board kV and cone-beam CT imaging, using the biopsy clip as a fiducial marker. Radiation was prescribed as described below and delivered in a single fraction. Within 10 days of radiation, lumpectomy and sentinel lymph node evaluation, if indicated, were completed in standard fashion. A negative margin of 2 mm was required. Post-operative conventional radiotherapy (46-50 Gy in 1.8-2.0 Gy/fraction) was administered to patients not satisfying eligibility criteria following surgical resection (n=3). Systemic therapy was prescribed at the discretion of the treating medical oncologist [Table 1].

TABLE 1
Characteristics of Patients Treated with
Preoperative Partial Breast Irradiation

| Characteristic | N (%) | |
|---|---|---|
| Age (years) | | |
| Median (range) | 66 | (55-78) |
| Race | | |
| White | 30 | (94) |
| Black | 2 | (6) |
| Body Mass Index | | |
| ≦25 | 7 | (22) |
| 25 < BMI < 30 | 14 | (44) |
| ≧30 | 11 | (34) |
| Histologic type | | |
| Ductal | 24 | (75) |
| Ductal carcinoma in situ (DCIS) | 7 | (22) |
| Invasive, other | 1 | (3) |
| Clinical Tumor Size* | | |
| DCIS | 7 | (22) |
| T1a | 4 | (13) |
| T1b | 14 | (44) |
| T1c | 7 | (22) |
| Pathologic Tumor Size | | |
| DCIS | | |
| <2 cm | 5 | (16) |
| ≧2 cm | 1 | (3) |
| T1a | 4 | (13) |
| T1b | 10 | (32) |
| T1c | 12 | (38) |
| Nodal status | | |
| N0 | 25 | (78) |
| N1 | 1 | (3) |
| Nx | 6 | (19) |
| Receptor Status | | |
| ER+/PR+ | 29 | (91) |
| ER+/PR− | 3 | (9) |
| Adjuvant Systemic Therapy | | |
| Endocrine therapy, alone | 23 | (72) |
| Endocrine and Chemotherapy | 2 | (6) |
| Neither Endocrine nor Chemotherapy | 7 | (22) |

*reflects the largest tumor diameter identified on physical exam or any breast imaging modality

### Study Endpoints

Patients were enrolled consecutively in cohorts of eight to a dose of 15 Gy, 18 Gy or 21 Gy in order to determine the maximum tolerated dose of preoperative partial breast radiotherapy. An additional eight patients were planned at the final dose level for further analysis of safety and efficacy. Patients were assessed 3-4 weeks after radiation for acute grade 3 or 4 toxicity. Any toxicity possibly, probably, or definitely related to radiation was considered dose-limiting (DLT). Escalation was prohibited if two or more DLTs were encountered in any eight patient dose cohort. In the event that no DLT was encountered, 21 Gy was defined a priori as the recommended phase II dose given the efficacy and limited toxicity seen at this dose in randomized trials evaluating intraoperative radiation 2,3. The National Cancer Institute's Common Terminology Criteria for Adverse Events (CTCAE), version 4.0, was used to score and grade acute and chronic treatment-related toxicity.

Cosmetic outcomes were assessed by both physician and patient at baseline and again at 6 months, 1, 2, and 3 years utilizing the NSABP B39/RTOG 0413 cosmesis evaluation scale.

### Radiation Response Imaging Analysis

When feasible between radiation treatment and surgical resection, patients underwent a second MRI in order to assess radiation response. Ktrans (volume transfer constant between blood plasma and extravascular-extracellular space), ve (extravascular-extracellular space fraction), semi-quantitative iAUC (initial area under contrast agent concentration curve) and ADC (apparent diffusion coefficient) were evaluated in patients with paired pre- and post-radiation MRI imaging (n=15). The Wilcoxon signed-rank test was used to assess the relative changes of each parameter in each area of interest (GTV, CTV and PTV) following radiotherapy. A correlation test was used to examine the linear dependence of relative parameter change on radiation dose. p<0.05 was considered statistically significant.

### Gene Expression Analysis

Formalin-fixed, paraffin-embedded (FFPE) pre- and post-treatment paired diagnostic biopsy and lumpectomy samples were used to assess changes in gene expression. FFPE RNA extraction and labeling was performed using the RNeasy FFPE kit from Qiagen, and the SensationPlus™ FFPE Amplification and Labeling Kit (Affymetrix, Inc. catalog #902312). All total RNA samples were assessed for quality using a NanoDrop ND8000 Spectrophotometer for absorbance ratios, and the Agilent Bioanalyzer 2100 for RIN scores. Whole transcriptome expression analysis was evaluated with HTA 2.0 arrays (Affymetrix, Inc. catalog #902162). Gene level expression estimates were obtained with Affymetrix Expression Console software (v.1.3) using RMA-Sketch workflow. Differential expression for paired samples was evaluated using the Bioconductor limma package with correction for multiple comparisons 16. Genes with FDR adjusted p-values (q-values) less than 0.05 were selected as differentially expressed in response to radiation. This set of genes was then tested for radiation dose effect using linear regression of the log2 fold change over dosage received. Those genes with regression p-values less than 0.0005 and q-values less than 0.25 were selected as having dose effect. Gene set analysis (GSA) was performed with the R package GSA using “two class paired” problem type and 100,000 permutations to estimate false discovery rates. The gene set collection used is “all GO (gene ontology) gene sets” which contains 1454 gene sets in total.

### Results

### Patients

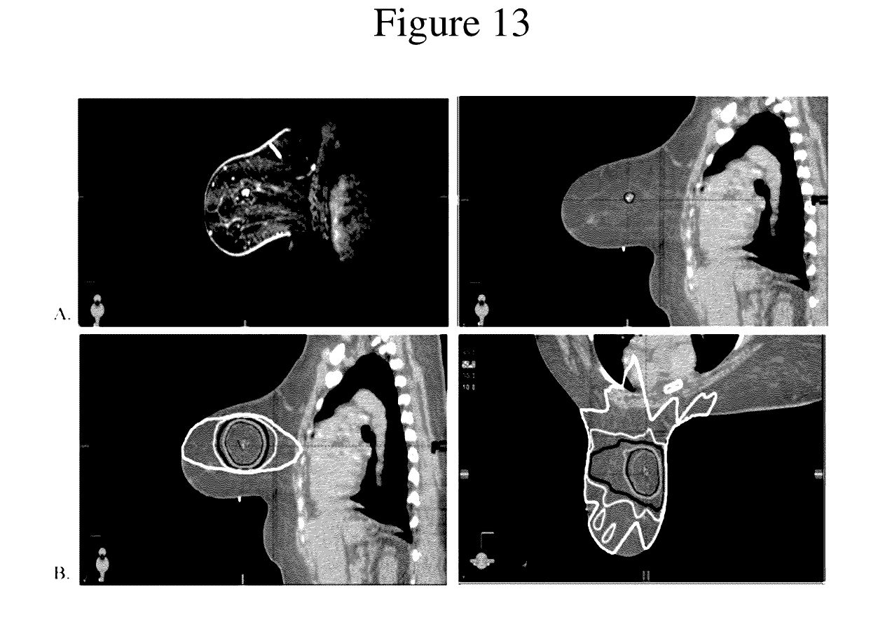

Between August 2010 and March 2013, 32 patients were enrolled [Table 1]. The median breast volume was 1590 cm3 (range 3293-3733) while the median target volume (CTV) was 43 cm3 (range 20-66). Due to the large discrepancy between target and normal breast tissue volumes, the median amount of breast receiving prescription dose was only 4% of the total breast volume. Furthermore, due to the highly conformal nature of the radiation techniques [FIG. 13], only 14% of the breast received half of the prescription dose. The mean heart dose was less than 0.1 Gy for the entire cohort and the highest maximum dose was 1 Gy. Radiation doses to the lungs, thyroid and brachial plexus were negligible. The median maximum skin dose (as a proportion of prescription dose: 77.5% (51.7-97.6%)) approached the prescribed dose in some patients. However, the median dose to a lcc skin volume fell to only 59.8% (41.1-84.2%) while the 10 cc median dose was only 37.7% (25.5-53.9%).

### Clinical Outcomes

Eight patients received 15 Gy, eight 18 Gy, and 16 received 21 Gy. No acute dose-limiting grade 3 or 4 radiation-related toxicities were seen. No wound dehiscence was observed. Median follow-up is 23 months (range 11-37; excluding one patient who died in a motor-vehicle accident at 2 months of follow-up). Toxicities attributed to radiation are reported in Table 2. Side effects were largely mild and consistent with expected sequelae of surgical and/or radiation therapy. Fibrosis occurred in 77% of patients; most were grade 1. Dermatitis and breast pain were also common.

According to study protocol, 3 patients received post-operative external beam radiotherapy. This was due in one case to DCIS extending over >2 cm, a positive lymph node in another, and a mixed ductal/metaplastic tumor in the final patient. Two grade 3 chronic toxicities were seen in a patient from this group who was diagnosed post-treatment with a connective tissue disorder. Another patient with diabetes developed a post-operative wound infection and all three had fair/poor cosmetic outcomes.

TABLE 2
(A)

| | Total | | Grade 1 | | Grade 2 | |
|---|---|---|---|---|---|---|
| CTCAE Toxicity Term | n | % | n | % | n | % |
| Breast pain | 7 | 23% | 5 | 16% | 2 | 6% |
| Dermatitis | 12 | 39% | 9 | 29% | 3 | 10% |
| Fatigue | 2 | 6% | 2 | 6% | 0 | 0% |
| Fibrosis | 7 | 23% | 7 | 23% | 0 | 0% |
| Infection | 1 | 3% | 0 | 0% | 1 | 3% |
| Seroma | 10 | 32% | 10 | 32% | 0 | 0% |
| Skin ulceration | 1 | 3% | 1 | 3% | 0 | 0% |

(B)

| | Total | | Grade 1 | | Grade 2 | | Grade 3 | |
|---|---|---|---|---|---|---|---|---|
| CTCAE Toxicity Term | n | % | n | % | n | % | n | % |
| Breast atrophy | 5 | 16% | 2 | 13% | 2 | 6% | 1 | 3% |
| Breast pain | 6 | 19% | 4 | 16% | 2 | 6% | 0 | 0% |
| Dermatitis | 6 | 19% | 5 | 3% | 1 | 3% | 0 | 0% |
| Fatigue | 1 | 3% | 1 | 58% | 0 | 0% | 0 | 0% |
| Fibrosis | 22 | 71% | 18 | 0% | 3 | 10% | 1 | 3% |
| Infection | 1 | 3% | 0 | 0% | 1 | 3% | 0 | 0% |
| Lymphodema | 1 | 3% | 0 | 3% | 1 | 3% | 0 | 0% |
| Prurirus | 1 | 3% | 1 | 6% | 0 | 0% | 0 | 0% |
| Seroma | 3 | 10% | 2 | 19% | 1 | 3% | 0 | 0% |
| Skin hyperpigmentation | 7 | 23% | 6 | 0% | 1 | 3% | 0 | 0% |
| Skin infection | 1 | 3% | 0 | 6% | 1 | 3% | 0 | 0% |
| Telangiectasia | 2 | 6% | 2 | 0% | 0 | 0% | 0 | 0% |

(A) Acute toxicity (90 days or less) possibly, probably, or definitely related to radiation.
(B) Chronic toxicity possibly, probably, or definitely related to radiation.
CTCAE: Common Terminology Criteria for Adverse Events.

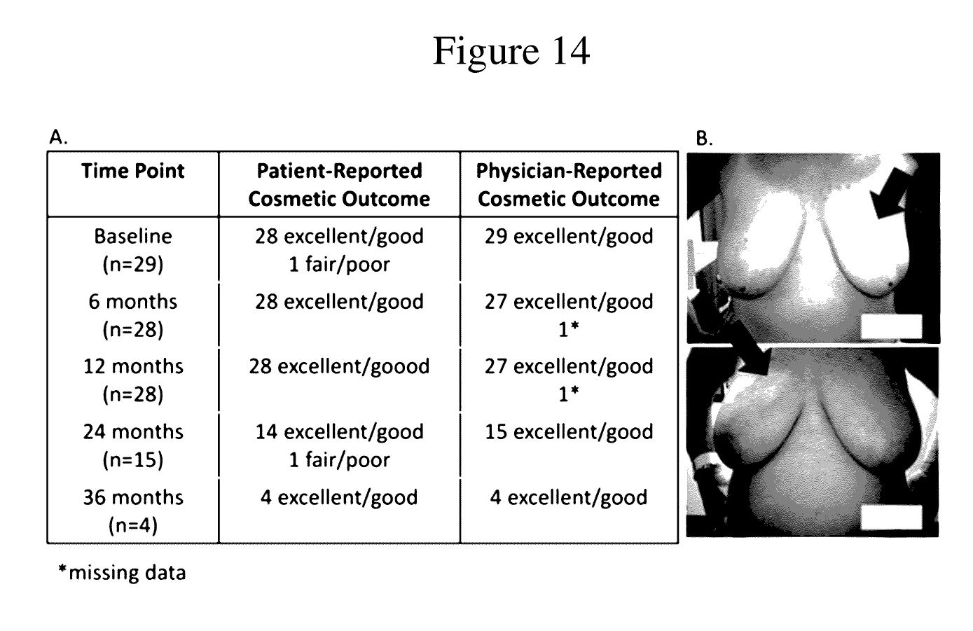

In contrast, all patients receiving preoperative therapy alone had good or excellent physician-reported cosmetic outcomes at each time point [FIG. 14]. All patients remain without evidence of disease.

### MRI Imaging and Radiation Response

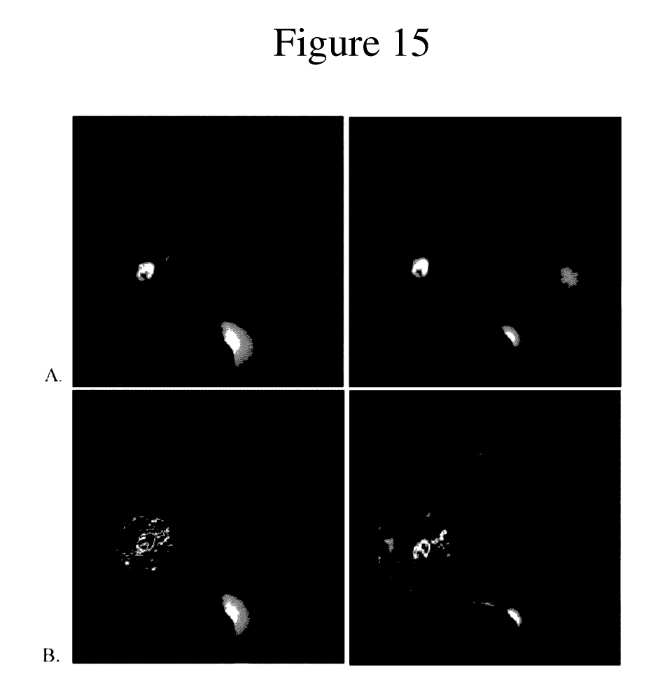

Fifteen of 32 patients had interpretable post-operative MR imaging. Among those who underwent both pre- and post-treatment MRI, the iAUC decreased significantly in the PTV (p<0.006) and CTV (p<0.006) volumes suggesting an increase in the post-radiation vascular permeability [FIG. 15]. In contrast, ve significantly increased in both the PTV and CTV (p<0.05) consistent with decreased cellular density. The impact of radiation appears to be dose-related with greater relative changes associated with increasing radiation dose in all parameters (rADC, iAUC 6 min, Ktrans, and ve) and nearly all areas of interest (GTV, CTV, PTV).

### Changes in Gene Expression After Radiation

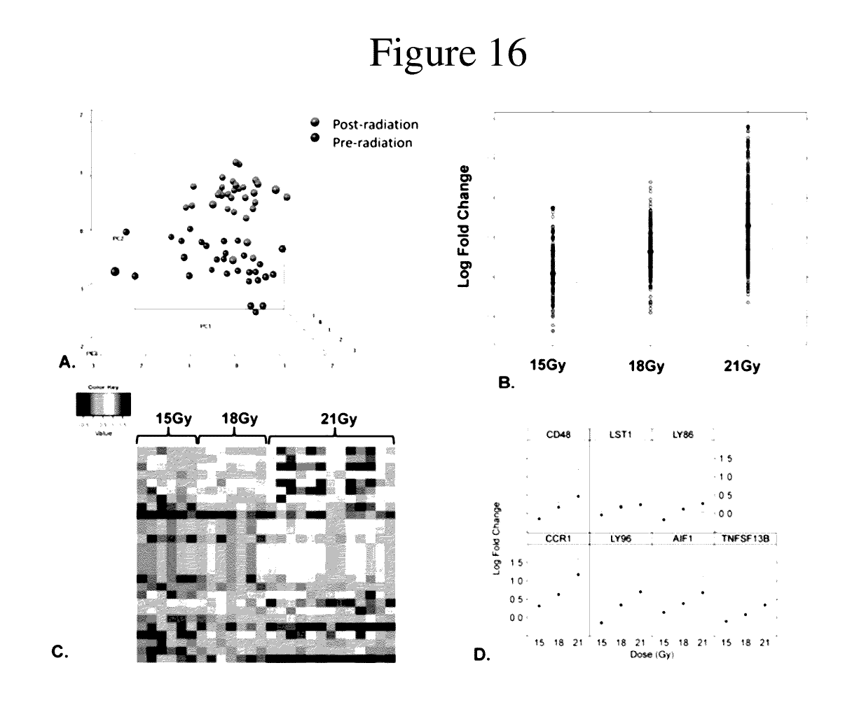

Twenty-six patients had pre- and post-operative FFPE tissue pairs for analysis of gene expression. Principal component analysis revealed marked separation of samples before and after radiation with minimal overlap [FIG. 16A]. After selecting genes whose expression levels varied with radiation, we were able to identify a subset of 27 genes with evidence of significant dose-dependent changes. Increasing dose appears to primarily induce rather than repress gene expression [FIG. 16B/C] and this cohort was enriched for modulators of the inflammatory and immune response such as CD48, LST1, LY86, LY96, AIF1, and CCR1 [FIG. 16D]. Specifically, increased expression of AIF1, or allograft inflammatory factor 1, has been observed in response to vascular trauma 17. The dose-dependent induction of AIF was conserved across multiple probe sets and is consistent with the MRI data described above suggesting early vascular trauma. CD48, a plasma membrane immunoglobulin, is intimately involved in immune signaling and has been reported to act as a regulator that inhibits the malignant transformation of stem cells 18. Finally, CCR1, a G-protein coupled plasma membrane receptor, is a known mediator of host immune response with a potential role in the induction of apoptotic cell death 19.

Gene set analysis utilizing all genes exhibiting significant change in response to radiation further identifies programmed cell death as a pathway of major significance in the radiation response of these tumors [Induction of Apoptosis By Intracellular Signals: p=4.00E-05, FDR 0.0418, Apoptosis Go: p=0.00388, FDR 0.0418; Programmed Cell Death: p=0.00416, FDR 0.0418; Supplemental Table 2].

### Discussion

External beam radiotherapy has emerged as the most accessible and thus, most common method of PBI delivery in the United States. Long-term toxicity and cosmesis results with this technique are conflicting but in the largest randomized report to date, suboptimal cosmetic outcomes were noted 11. Though the cause has not been clearly identified, multiple reports have implicated generous post-operative treatment volumes in this process 20. The size of the post-operative seroma relative to the actual tumor size makes it unlikely that large and consistent reductions could be achieved post-operatively to address this issue. Target volumes in the post-operative setting typically range from 9-26% of the whole breast receiving prescription dose and 34-49% 20 receiving half of prescription dose. This is due, in part, to the larger margins required to account for geographic target uncertainty post-resection and daily reproducibility with fractionated radiation in the supine position.

In contrast, preoperative targeting of the intact tumor with single-fraction delivery allows for treatment of a well-circumscribed, dramatically reduced target volume. In our study, identification of the tumor as a target was feasible and resulted in only 4% of the whole breast receiving prescription dose and 14% receiving half of prescription dose. With short follow-up, our single-fraction approach was well-tolerated despite skin doses that routinely approached the dose prescribed. No patient experienced acute dose-limiting toxicity up to 21 Gy. Furthermore, chronic toxicity and cosmetic outcomes at 2 years appear comparable to standard whole-breast treatment.

Long-term follow-up with larger patient cohorts will be needed to determine if this approach has comparable safety and efficacy to fractionated radiation. However, the precedent exists for the single-dose approach with multiple intra-operative series demonstrating high rates of local control. Two large randomized European equivalence trials (ELIOT, TARGIT A) have compared intra-operative radiation only to whole breast treatment2,3 in a more diverse patient population than included in this study. Both trials found a statistically significant reduction in local recurrence for patients receiving whole breast versus intraoperative treatment (5 year ipsilateral breast tumor recurrence rate (IBTR) of 4.4% with 0.4% in ELIOT; 4-year IBTR 3.3% versus 1.3% in TARGIT-A), but the small absolute differences in both studies are unlikely to be clinically meaningful in the context of careful patient selection and low overall recurrence rates seen in the modern era.

The use of a targeted preoperative approach has been explored by other investigators. Researchers at the University of Maryland enrolled women with early stage breast cancer on a clinical trial evaluating the impact of preoperative partial breast irradiation on pathologic complete response (NCT01014715). Bondiau et al. conducted a phase I study testing 5 dose levels of focused radiotherapy delivered during preoperative chemotherapy in high-risk patients that may not otherwise have been candidates for breast conservation 21. Surgery and conventional post-operative radiotherapy followed neoadjuvant chemoradiotherapy. Total dose ranged from 19.5 Gy to 31.5 Gy (10.5 Gy/fraction). Only 1 grade 3 or greater treatment-related toxicity was measured over a period of 8 months. This is in contrast to the fair/poor cosmetic outcomes noted in our patient cohort when preoperative and postoperative radiotherapy was combined, despite presumably smaller target volumes. However, comorbid conditions in our group would have also increased the risk of complications associated with conventional therapy. Alternatively, it may be that the fractionation of the preoperative dose in the French series helped to decrease complication rates.

One especially exciting aspect of the preoperative approach is the potential to enhance our understanding of breast cancer radiation biology. Our MRI findings suggest that vascular permeability is increased and cellular density decreased. In concert with gene expression findings suggesting initiation of inflammatory/immune response and programmed cell death, radiation appears to induce a vigorous response that may be partially mediated through host immunity. If confirmed in larger cohorts and tied to clinical outcomes in future studies, these genes have the potential to act as radiation response biomarkers as well as therapeutic targets to enhance radiation sensitivity in more resistant tumors 22. Furthermore, these data show the power of integrating functional imaging and gene expression in the assessment of tumor response to radiation 23,24.

Our preliminary data suggests that though the preoperative approach is feasible, well-tolerated and generates novel and interesting correlative data, it is not without limitations. The number of patients treated in this phase I trial is small and the follow-up is still short. Clinical outcomes will need to be verified in a larger patient cohort with many years of follow-up. Furthermore, the specifics of this technique may benefit from further optimization. For example, we anticipate that the small treatment volumes in this study will reduce long-term complications. However, the large single-dose may offset the benefits of volume reduction long-term. A fractionated preoperative approach may provide a more optimal complement to the small target volume. In addition, larger treatment margins may be required to sterilize all microscopic disease. Modern studies correlating MRI and pathology findings suggest that for many cases there is general agreement to within 0.5 cm. Overestimation remains common (33%) but the most worrisome finding for a preoperative technique, underestimation of tumors, was noted in only 15%12. In another study, 93% of patients had no invasive disease more than 10 mm's beyond that identified on MRI25. We used a generous margin in this series, but at present, the optimal margin has not been established. In future studies, increases in the margin size would be feasible without approaching post-operative volumes.

### Conclusion

This is the first report of an innovative, preoperative approach to PBI, which demonstrates clear feasibility in women with low risk, early stage breast cancer. The technique captures the appeal of a single-dose approach without incurring additional equipment costs or logistic complexities. Importantly, this approach also improves upon the inaccuracies and large treatment volume requirements of post-operative targeting. Short-term outcomes have been favorable, with toxicities at lower or expected rates than those reported with conventional external beam radiotherapy. Tumor response to radiation is marked by early radiologic and genomic indicators of immune response and cell death.

This study is a first and hypothesis-generating step towards a transformative PBI approach. Preoperative radiation should be tested in future clinical trials as it has the potential to challenge the current treatment paradigm and provide a path forward to identify radiation response biomarkers. Such efforts will inform the design of the next generation of trials evaluating preoperative radiation and biologically driven radiation therapy.

### REFERENCES FOR EXAMPLE 2

1. Smith B D, Arthur D W, Buchholz T A, et al. Accelerated partial breast irradiation consensus statement from the American Society for Radiation Oncology (ASTRO). International journal of radiation oncology, biology, physics. Jul 15; 2009 74(4):987-1001.
2. Vaidya J S, Wenz F, Bulsara M, et al. Risk-adapted targeted intraoperative radiotherapy versus whole-breast radiotherapy for breast cancer: 5-year results for local control and overall survival from the TARGIT-A randomised trial. Lancet. Feb. 15; 2014 383(9917):603-613. [PubMed: 24224997]
3. Veronesi U, Orecchia R, Maisonneuve P, et al. Intraoperative radiotherapy versus external radiotherapy for early breast cancer (ELIOT): a randomised controlled equivalence trial. The lancet oncology. December; 2013 14(13):1269-1277. [PubMed: 24225155]
4. Julian T B, Costantino J P, Vicini F A, et al. Early toxicity results with 3D conformal external beam therapy (CEBT) from the NSABP B-39/RTOG 0413 accelerated partial breast irradiation (APBI) trial. Journal of clinical oncology: official journal of the American Society of Clinical Oncology. 2011; 29(suppl; abstr 1011) suppl; abstr 1011.
5. Hepel J T, Tokita M, MacAusland S G, et al. Toxicity of three-dimensional conformal radiotherapy for accelerated partial breast irradiation. International journal of radiation oncology, biology, physics. Dec. 1; 2009 75(5):1290-1296.
6. Jagsi R, Ben-David M A, Moran J M, et al. Unacceptable cosmesis in a protocol investigating intensity-modulated radiotherapy with active breathing control for accelerated partial-breast irradiation. International journal of radiation oncology, biology, physics. Jan. 1; 2010 76(1):71-78.
7. Formenti S C, Hsu H, Fenton-Kerimian M, et al. Prone accelerated partial breast irradiation after breast-conserving surgery: five-year results of 100 patients. International journal of radiation oncology, biology, physics. Nov. 1; 2012 84(3):606-611.
8. Rodriguez N, Sanz X, Dengra J, et al. Five-year outcomes, cosmesis, and toxicity with 3-dimensional conformal external beam radiation therapy to deliver accelerated partial breast irradiation. International journal of radiation oncology, biology, physics. Dec. 1; 2013 87(5):1051-1057.
9. Shah C, Wilkinson J B, Lanni T, et al. Five-year outcomes and toxicities using 3-dimensional conformal external beam radiation therapy to deliver accelerated partial breast irradiation. Clinical breast cancer. June; 2013 13(3):206-211. [PubMed: 23103365]
10. Mozsa E, Meszaros N, Major T, et al. Accelerated partial breast irradiation with external beam three-dimensional conformal radiotherapy. Five-year results of a prospective phase II clinical study. Strahlentherapie and Onkologie: Organ der Deutschen Rontgengesellschaft . . . [et al]. May; 2014 190(5):444-450.
11. Olivotto I A, Whelan T J, Parpia S, et al. Interim cosmetic and toxicity results from RAPID: a randomized trial of accelerated partial breast irradiation using three-dimensional conformal external beam radiation therapy. Journal of clinical oncology: official journal of the American Society of Clinical Oncology. Nov 10; 2013 31(32):4038-4045. [PubMed: 23835717]12. Grimsby G M, Gray R, Dueck A, et al. Is there concordance of invasive breast cancer pathologic tumor size with magnetic resonance imaging? American journal of surgery. October; 2009 198(4):500-504. [PubMed: 19800455]
13. XXXX.
14. Timmerman R D. An overview of hypofractionation and introduction to this issue of seminars in radiation oncology. Seminars in radiation oncology. October; 2008 18(4):215-222. [PubMed: 18725106]
15. XXXX.
16. Gentleman R C, Carey V J, Bates D M, et al. Bioconductor: open software development for computational biology and bioinformatics. Genome biology. 2004; 5(10):R80. [PubMed: 15461798]
17. Autieri M V. cDNA cloning of human allograft inflammatory factor-1: tissue distribution, cytokine induction, and mRNA expression in injured rat carotid arteries. Biochemical and biophysical research communications. Nov. 1; 1996 228(1):29-37. [PubMed: 8912632]
18. Boles N C, Lin K K, Lukov G L, Bowman T V, Baldridge M T, Goodell M A. CD48 on hematopoietic progenitors regulates stem cells and suppresses tumor formation. Blood. Jul. 7; 2011 118(1):80-87. [PubMed: 21576698]
19. Zeng H Y, Lu Q J, Liu Q, Liu K G, Wang N L. The role of CCR1 expression in the retinal degeneration in rd mice. Current eye research. March; 2011 36(3):264-269. [PubMed: 21275605]
20. Lei R Y, Leonard C E, Howell K T, et al. Four-year clinical update from a prospective trial of accelerated partial breast intensity-modulated radiotherapy (APBIMRT). Breast cancer research and treatment. July; 2013 140(1):119-133. [PubMed: 23824363]
21. Bondiau P Y, Courdi A, Bahadoran P, et al. Phase 1 clinical trial of stereotactic body radiation therapy concomitant with neoadjuvant chemotherapy for breast cancer. International journal of radiation oncology, biology, physics. Apr. 1; 2013 85(5):1193-1199.
22. Formenti S C, Demaria S. Combining radiotherapy and cancer immunotherapy: a paradigm shift. Journal of the National Cancer Institute. Feb. 20; 2013 105(4):256-265. [PubMed: 23291374]
23. Dewhirst M W, Chi J T. Understanding the tumor microenvironment and radioresistance by combining functional imaging with global gene expression. Seminars in radiation oncology. October; 2013 23(4):296-305. [PubMed: 24012344]
24. Chi J T, Thrall D E, Jiang C, et al. Comparison of genomics and functional imaging from canine sarcomas treated with thermoradiotherapy predicts therapeutic response and identifies combination therapeutics. Clinical cancer research: an official journal of the American Association for Cancer Research. Apr. 15; 2011 17(8):2549-2560. [PubMed: 21292819]
25. Schmitz A C, van den Bosch M A, Loo C E, et al. Precise correlation between MRI and histopathology—exploring treatment margins for MRI-guided localized breast cancer therapy. Radiotherapy and oncology: journal of the European Society for Therapeutic Radiology and Oncology. November; 2010 97(2):225-232. [PubMed: 20826026]

### Example 3

### FAS and TNFRSF10B

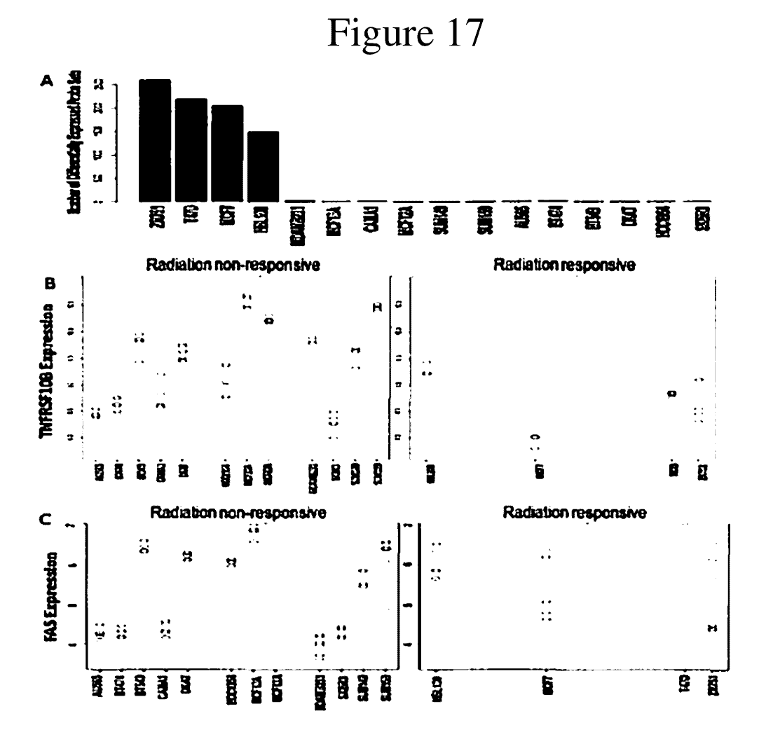

FIG. 17 shows the differential gene expression response to radiation in a panel of 16 breast cancer cell lines. The effect of radiation on the expression levels of TNFRSF10B and FAS is significantly different in the responsive vs. non responsive cell lines. (Black circles are pre-RT and red circles are postRT).

We validated the differential expression of FAS and TNFRSF10B using Western blotting and qPCR assays. We then went on to evaluate whether FAS was necessary and sufficient for radiation response. We knocked out FAS in a radiation responsive cell line (shFAS/knockdown MCF7 cell line) and observed increased radiation resistance. Conversely, we activated FAS and TNFRSF10B receptors in our most non-responsive breast cancer cell line (SUM159) and observed increased radiation sensitivity (CH 11, a FAS agonistic antibody was obtained from Millipore, cat #05-201; the TNFRSF10B agonistic antibody was obtained from R&D systems, cat #MAB63 1). In addition, Western blot analysis revealed that in the SUM159 breast cancer cell lines these activating antibodies significantly induced apoptosis proteases caspase-9 and caspase-3, which is a sign of activating the apoptosis pathway. The pro-apoptotic effects following the binding of these agonistic antibodies to the FAS and TNFRSF10B/DR5 receptor are in accordance with other studies. Our animal studies for evaluating FAS and TNFRSF10B/DRS modulation are ongoing.

Our results for the modulation of TNFRSF10B/DR5 on radiation sensitivity has not been published yet, but the FAS results is presented in our Radiation Research manuscript as presented below: Though our results indicated that FAS induction appeared to be differentially induced in the more favorable, luminal-type breast cancers, it was not clear that this could then be utilized as a tool to modify radiation response. Therefore, we selected one cell line from the responsive cohort (luminal: MCF7) and one from the non-responsive cohort (basal: SUM 159) to determine if sensitivity to radiation would change as a result of FAS modulation.

We selected the radiation responsive MCF7 cell line to determine if radiation sensitivity could be reversed in the absence of FAS expression. We first verified that shRNA effectively knocked down the FAS receptor using western blotting. Radiation was also applied and no FAS induction was seen in the knockdown. Clonogenic cell survival assays were then conducted and compared to the control shRNA MCF7 cell line with intact FAS. Both cell lines were equally radiosensitive at 4 Gy, but increased radioresistance was observed with FAS knockdown for the lower doses of I and 2 Gy. In contrast, when FAS was activated by FAS stimulating antibody CH 11, the non-radiation responsive SUM 159 demonstrated significantly greater radiation sensitivity. The dose to achieve a surviving fraction of 0.1 was approximately 8 Gy in the control cells and 3.25 Gy in the CHI 1 treated SUM159 cells, yielding a dose modifying factor of 2.5. This occurred despite a lack of visible FAS induction.

1. A method comprising:
i) obtaining a tumor sample from a subject,
ii) measuring the expression level of no more than 25 biomarkers in the sample, wherein the biomarkers comprise at least one biomarker selected from FAS, TNFRSF10B, CD48, LST1, LY86, CCR1, LY96, AIF1, TNFSF13B, TP5313 (p53-inducible protein 3) and caspase 9.

2. The method of claim 1, wherein the expressions levels of no more than 10 biomarkers are measured.

3. The method of any claim 1, wherein the biomarkers comprise FAS, TNFRSF10B, or both.

4. The method of claim 1, further comprising administering radiation therapy to the subject.

5. The method of claim 4, wherein the radiation therapy is administered to the subject prior to obtaining the tumor sample from the subject.

6. The method of claim 4, further comprising obtaining a tumor sample before the administering the radiation therapy.

7. The method of claim 1, wherein the expression level of the biomarker is the mRNA expression level.

8. The method of claim 7, wherein the biomarker is measured using RT-PCR.

9. The method of claim 1, wherein the tumor sample is a breast cancer tumor.

10. A method of treating cancer in a subject comprising administering to the subject radiation therapy based on the expression level of no more than 25 biomarkers in a tumor sample from the subject, wherein the biomarkers comprise at least one biomarker selected from FAS, TNFRSF10B, CD48, LST1, LY86, CCR1, LY96, AIF1, TNFSF13B, TP5313 (p53-inducible protein 3) and caspase 9.

11. The method of claim 10, wherein the expressions levels of no more than 10 biomarkers are measured.

12. The method of claim 10, wherein the biomarkers comprise FAS, TNFRSF10B, or both.

13. The method of claim 10, wherein the expression level of the biomarker is the mRNA expression level.

14. The method of claim 13, wherein the biomarker is measured using RT-PCR.

15. The method of claim 10, wherein the cancer is breast cancer and the tumor sample is a breast cancer tumor.

16. The method of claims 10, further comprising measuring the expression level of the no more than 25 biomarkers in a second tumor sample from the subject after administration of the radiation therapy.

17. (canceled)

18. (canceled)

19. (canceled)

20. A method of treating cancer in a subject comprising administering a therapeutically effective amount of a FAS agonist and/or a TNFRSF10B agonist to the subject, and administering radiation therapy to the subject.

21. The method of claim 20, wherein the FAS agonist and/or a TNFRSF10B agonist are administered intratumorally.

22. The method of claim 20, wherein the cancer is breast cancer.

23. The method of claim 2022, wherein the FAS agonist and/or a TNFRSF10B agonist comprises an antibody.

24. (canceled)
